(19) United States
(12) Patent Application Publication
(10) Pub. No.: US 2023/0200712 A1
(43) Pub. Date: Jun. 29, 2023

(54) SYSTEMS AND METHODS FOR DETECTING THE PRESENCE OF ELECTRICALLY EVOKED COMPOUND ACTION POTENTIALS (eCAPS), ESTIMATING SURVIVAL OF AUDITORY NERVE FIBERS, AND DETERMINING EFFECTS OF ADVANCED AGE ON THE ELECTRODE-NEURON INTERFACE IN COCHLEAR IMPLANT USERS

(71) Applicant: Ohio State Innovation Foundation, Columbus, OH (US)

(72) Inventors: Jeffrey SKIDMORE, Galloway, OH (US); Shuman HE, Columbus, OH (US); Xia NING, Dublin, OH (US)

(21) Appl. No.: 17/927,309

(22) PCT Filed: May 21, 2021
(86) PCT No.: PCT/US2021/033604

§ 371 Date: Nov. 22, 2022

Related U.S. Application Data
(60) Provisional application No. 63/182,402, filed on Apr. 30, 2021.
(60) Provisional application No. 63/143,689, filed on Jan. 29, 2021.
(60) Provisional application No. 63/028,677, filed on May 22, 2020.

(51) Int. Cl. A61B 5/38; A61B 5/12; A61B 5/00

(52) U.S. Cl. CPC A61B 5/38; A61B 5/125; A61B 5/686; A61B 5/7246; A61B 5/4047

(57) ABSTRACT

Disclosed herein are of systems, methods, and computer-program products for determining if a response is an electrically evoked compound action potential (eCAP), refining raw data of an eCAP amplitude growth function (AGF) and utilizing maximum (i.e., steepest) slope from moving linear regression to effectively estimate cochlear nerve function, and determining quality of an electrode-neuron interface (ENI) using a model developed from eCAP attributes.

### CROSS REFERENCE TO RELATED APPLICATIONS

This application claims priority to and benefit of U.S. provisional patent application Ser. No. 63/028,677 filed May 22, 2020; U.S. provisional patent application Ser. No. 63/143,689 filed Jan. 29, 2021; and, U.S. provisional patent application Ser. No. 63/182,402 filed Apr. 30, 2021, each of which are fully incorporated by reference and made a part hereof.

### GOVERNMENT SUPPORT CLAUSE

This invention was made with government support under grant/contract numbers DC017846 and DC016038 awarded by the National Institutes of Health. The government has certain rights in the invention.

### BACKGROUND

As shown in FIG. 1, cochlear implants convert sounds (picked up by a microphone) into electrical representations (in the sound processor) which are transmitted through the head and stimulate nerves surrounding the cochlea. Cochlear implants bypass the normal hearing pathway (i.e. middle ear bones and inner ear hair cells), which is usually not functional when somebody is deaf. However, cochlear implants still rely on the cochlear nerve to be functional in order to carry the signal to the brain for further processing and interpretation (as shown in FIG. 2).

In humans, there is no way to directly evaluate how well the cochlear nerve functions. However, as shown in FIG. 3, user defined stimuli can be sent through one electrode to stimulate the surrounding neurons. The neurons then respond and the electrical response can be recorded by a neighboring electrode. The neural response is called the electrically-evoked compound action potential (eCAP). The eCAP response can be characterized as an indirect measure of neural function.

However, a challenge for clinical application of eCAP technology is identifying the presence of an eCAP instead of measurement noise. Identifying eCAPs is typically done visually by a highly trained researcher and/or audiologist. This takes considerable amount of time and training, which prevents the clinical application of objective eCAP measures.

Therefore, systems and methods are desired that overcome challenges in the art, some of which are described above. More specifically, there is a need for systems and methods to automatically detecting the presence of electrically evoked compound action potentials (eCAPs) in cochlear implant patients.

Furthermore, the most used parameter to characterize the response is the slope of the eCAP amplitude growth function (AGF). The AGF is recorded by measuring the eCAP response amplitude voltage (y-axis), with increasing stimulation levels/current (x-axis), as shown in FIG. 4, which shows AGF data for an anesthetized animal that resembles a sigmoidal function. The slope of the eCAP AGF has been shown to be strongly correlated with the number of surviving auditory neurons in animals (Ramekers et al., 2014; Pfingst et al., 2015; Pfingst et al., 2017, each of which are incorporated by reference).

There are three general categories of AGF data of interest (see FIG. 5): Anesthetized animals, awake animals, and awake humans. Anesthetized animals can be stimulated at high enough levels that the function saturates (i.e. reaches a plateau). These AGFs are easily fit with a sigmoidal function (s-shape function with a plateau curving to a linear region, curving to another plateau), and the slope is easily obtained. The slope is defined as the line tangent to the sigmoidal fit at the inflection point (level 50% in the left figure in FIG. 5). See Ramekers et al., 2014, incorporated by reference, detailing the sigmoidal function (Equation 1) and slope calculation.

For awake animal and awake humans (FIG. 5, center and right figures), saturation of the AGF is rarely obtained because it is not possible to stimulate at high enough levels to reach the upper plateau. This is due to subject/patient discomfort and/or exceeds the stimulation capabilities (i.e. voltage level) of the implant device. Therefore, the AGF usually resembles a ‘partial sigmoid.’ Moreover, these datasets are not as ‘clean’ as for the anesthetized animals, meaning there is a lot more ‘noise’ or measurement error/variability.

Researchers and cochlear implant manufacturers have been trying to quantify the slope of the eCAP AGF in humans (e.g., Brown et al., 1990; Kim et al, 2010; He et al., 2018, Schvartz-Leyzac and Pfingst, 2016, 2018, each of which are incorporated by reference). Because the sigmoidal fitting does not work well for these datasets, different research groups and cochlear implant manufacturers/suppliers use different approaches to quantify the eCAP AGF slope. Due to the well-known limitations of sigmoidal and linear regression in calculating the slope, most researchers now create custom methods for their specific data set. This involves the expert researcher manually looking at the raw data, deciding which data points to exclude, and then fitting a line with linear regression. This is not translatable to clinical care because it requires an expert user and is not automated. Some groups make blanket criteria to exclude data points (e.g., any value less than 100 uV). However, these techniques only work for the specific dataset being analyzed. They do not apply generally to all AGFs, including multiple patient populations with different neural response characteristics (e.g., children and adults).

Currently; however, there is no standard way to quantify the slope in humans. Therefore, researchers and cochlear implant manufacturers/providers calculate the slope in different ways. However, each current method used to quantify the slope has limitations. Therefore, systems and methods are desired that overcome challenges in the art, some of which are described above. There is a need for systems and methods to quantify the slope of the eCAP AGF that addresses all of these limitations and can work for any eCAP AGF of all animals, whether human (adults and children) or non-human.

Finally, older cochlear implant (CI) patients generally have worse speech perception 62 capabilities than younger CI patients (e.g., Sladen & Zappler, 2005; Lenarz et al., 2012; 63 Lin et al., 2012; Roberts et al., 2013). While declining cognitive function with advancing age may contribute to these speech perception deficits in older CI patients (Budenz et al., 2011; Lin et al., 2011, 2012), poor speech perception has also been attributed to deteriorations in the auditory system (Friedland et al., 2010; Roberts et al., 2013). The mechanisms underlying speech perception deficits in older CI patients still remain unclear, which creates challenges in providing clinical care for these patients. As a step toward understanding the neurophysiological mechanisms underlying speech perception deficits in older CI patients, the effects of advanced age on how effectively a CI electrode stimulates the targeted cochlear nerve (CN) fibers (i.e., the electrode-neuron interface [ENI]) (Bierer, 2010) is examined.

Multiple factors affect the quality of the ENI, including the position of the electrode array within the cochlea, the impedances of intracochlear tissues, and the number and responsiveness of CN fibers (Bierer, 2010). Electrical stimuli are delivered by the CI to nearby CN fibers which then encode and transmit the information to higher-level neural structures for further processing and interpretation. Therefore, the quality of the ENI should theoretically be an important factor for speech perception. Numerous studies provide results that support this theory (e.g., Kim et al., 2010; Kirby et al., 2010, 2012; Garadat et al., 2013; Long et al., 2014; Pfingst et al., 2015; He et al., 2018; Skidmore et al., 2021a).

In CI users, the quality of the ENI can be assessed using neurophysiological measures of the eCAP. Like the ENI, neurophysiological measures of the eCAP are affected by electrode position, intracochlear resistance, and CN fiber density (e.g., Eisen & Franck 2004; Shepherd et al., 2004; Brown et al., 2010; Ramekers et al. 2014; Schvartz-Leyzac & Pfingst, 2016; Pfingst et al. 2015, 2017; He et al., 2018; Schvartz-Leyzac et al., 2020). Specifically, animals with higher densities of spiral ganglion neurons (SGNs) tend to have shorter refractory times, larger eCAP amplitudes, and larger slopes of eCAP AGFs than animals with fewer functional SGNs (Shepherd et al., 2004; Ramekers et al. 2014; Pfingst et al. 2015, 2017). In humans, eCAP thresholds and the slopes of eCAP AGFs have been shown to be affected by electrode position and intracochlear resistance (e.g., Young & Grohne, 2001; Eisen & Franck, 2004; Brown et al., 2010; Schvartz-Leyzac & Pfingst, 2016; Schvartz-Leyzac et al., 2020). Additionally, children with small or absent CNs present in imaging results (i.e., children with cochlear deficiency [CND]) have longer refractory times, smaller eCAP amplitudes, higher eCAP thresholds, and smaller eCAP AGF slopes compared to age-matched children with normal-sized CNs (He et al., 2018). Therefore, eCAP measures can be considered as a functional readout for the quality of the ENI.

eCAPs have been used to compare the quality of the ENI between pediatric and adult CI users. Human eCAP data suggests that the interface between CI electrodes and the target CN fibers differs between children and adults with CIs. Specifically, multiple studies have demonstrated that children and young adults have larger eCAP AGF slopes than older CI patients (Hughes et al., 2001; Cafarelli Dees et al., 2005; Brown et al., 2010; 107 Jahn & Arenberg, 2020). However, these differences in age groups may be reflective of differences in etiology between patients who are pre-lingually deafened vs post-lingually deafened (Bodmer et al., 2007; Brown et al., 2010; Zarowski et al., 2020). To date, the effect of advancing age on the quality of the ENI in post-lingually deafened adult CI patients has not been well established.

Therefore, systems and methods are desired that overcome challenges in the art, some of which are described above. There is a need for systems and methods to quantify the quality of the local (i.e., electrode-specific) ENI, and assess the effects of advanced age on the local ENI in CI patients.

### SUMMARY

Disclosed and described herein are systems and methods to address the above-described challenges. In particular, systems and methods are disclosed to automatically detect the presence of eCAPs in cochlear implant patients. As noted above, the eCAP is a measure of the responses of auditory nerve fibers that can recorded directly from a cochlear implant. A challenge for clinical application of eCAP technology is identifying the presence of an eCAP instead of measurement noise. The disclosed systems and methods automatically discern whether a measured value comprises an eCAP or is noise. In broad terms, the method compares neural response waveforms with a template eCAP waveform to determine if an eCAP exists or not in the neural response. Systems are also disclosed to implement the methods

Further, systems and methods are disclosed to quantify the slope of neural response functions. The disclosed systems and methods have several advantages including enabling individualized clinical care to improve hearing capabilities for existing cochlear implant users [including selection and/or adjustment (manual and/or automated) of the cochlear implant on a patient-specific basis]; and standardization by use of the disclosed methods, which can be used by all researchers to easily compare results across studies. Disclosed herein are embodiments of a system and a method of refining raw data of an electrically evoked compound action potential (eCAP) amplitude growth function (AGF) and utilizing maximum (i.e., steepest) slope from moving linear regression to effectively estimate cochlear nerve function. The disclosed methods provide an appropriate estimate for the slope for any raw AGF and correlates with an estimated number of surviving neurons in the cochlear nerve. Once the estimated number of surviving neurons in the cochlear nerve are determined, this information can be used to provide patients with a better clinical experience including design, selection and/or specification of the cochlear implant as well as adjustment of the cochlear implant (which can be manual and/or automated).

One aspect of the method to quantify the slope of neural response functions includes 1) receiving raw data comprised of a plurality of data points of AGF data (i.e., x,y pairs of stimulation level and eCAP amplitude); 2) resampling the raw data into a plurality (e.g., 100) of linearly spaced data points of AGF data; 3) perform linear regression on a moving window comprised of a subset (N) of the plurality of linearly spaced data points of AGF data (e.g., N=50 points), 3)(a), perform linear regression on a first window of comprised of data points 1 to N of the plurality of linearly spaced data points of AGF data (e.g., linear regression on data points [1-50] of the linearly spaced data points of AGF data) to determine a slope of the first window, 3)(b) move the window by one point (the subset is still comprised of the same number of data points) to form a second window and perform linear regression on data points 2 to N+1 (e.g., perform linear regression on data points [2-51]) to determine a slope of this second window, 3)(c) continue to perform linear regression on the data points of the plurality of linearly spaced data points of AGF data using the same size subset (N) of data points until the end of the plurality of linearly spaced data points of AGF data is reached (e.g., perform linear regression on data points [50-100]) to determine a slope of each of the plurality of different moving windows; 4) determine the steepest (i.e., maximum) slope among the slopes calculated in the plurality of different windows; and 5) correlate the selected steepest slope with surviving neurons in the cochlear nerve.

Finally, systems and methods are disclosed to estimate the quality of the electrode neuron interface (ENI) (at individual electrode locations in cochlear implant (CI) users. The quality of the ENI is significantly affected by the advanced age of CI patients. This aging effect appears be stronger in the basal region than in the middle and basal regions of the cochlea. However, this result is likely due to the combined effect of advanced age and etiologies that cause sensorineural hearing loss.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems.

FIG. 1 is an image illustrating an example of a cochlear implant.

FIG. 2 is an image illustrating how cochlear implants still rely on the cochlear nerve to be functional in order to carry the signal to the brain for further processing and interpretation.

FIG. 3 is an illustration of how user defined stimuli can be sent through one electrode of a cochlear implant to stimulate the surrounding neurons, with the compound neural response (i.e., eCAP) being recorded by a different electrode of the cochlear implant.

FIG. 4 illustrates an exemplary amplitude growth function (AGF) that is recorded by measuring the eCAP response amplitude voltage (y-axis), with increasing stimulation levels/current (x-axis), which resembles a sigmoidal function.

FIG. 5 is an illustration of three general categories of AGF data of interest.

FIG. 6A illustrates an exemplary flowchart for a method of determining if an eCAP exists or not in a neural response.

FIG. 6B illustrates an example of a template eCAP waveform.

FIG. 6C illustrates an example of a comparison of the recorded neural response and the re-sampled neural response.

FIG. 6D shows the recorded neural response and the re-sampled neural response after the mean voltages recorded in the a first time period (e.g., 600 μ-sec) are subtracted from the template and neural response waveforms.

FIG. 6E illustrates an example of scaling the template waveform vertically (voltage) to match the neural response waveform.

FIG. 6F illustrates an example of scaling the template waveform horizontally (time) to match the neural response waveform.

FIG. 6G illustrates an example of trimming the waveforms to the time period that overlaps after scaling.

FIG. 6H illustrates an example of re-sampling the waveforms at the same time periods as each other with higher resolution sampling occurring before the first time period (e.g., 600 μ-sec) to place emphasis on the first part of the waveforms in the correlation analysis.

FIG. 6I illustrates an example of calculating a correlation between the template and neural response waveform.

FIG. 6J illustrates an example correlation where the neural response is an eCAP (e.g., correlation value greater than 0.6).

FIG. 6K illustrates an example correlation where the neural response is not an eCAP (e.g., less than 0.6 correlation value).

FIG. 7 illustrates an exemplary flowchart for a method of providing an estimate for the slope of a neural response function characterized by a series of stimulation levels (i.e., input data) and corresponding amplitude of neural responses (i.e., output data).

FIG. 8 illustrates some of the steps of a method of providing an estimate for the slope of a neural response function characterized by a series of stimulation levels (i.e., input data) and corresponding amplitude of neural responses (i.e., output data).

FIGS. 9A-9P illustrate comparisons of calculating the slope of the AGF data using the disclosed embodiments and conventional methods of calculating the slope, in both animals and humans.

FIG. 10, upper panels, illustrate eCAP waveforms measured at different masker-probe intervals (MPIs) for stimulating electrode 3 in one adult younger than 68 years (A08), and one adult older than 68 years (A54). Waveforms are arranged based on MPI duration (in ms), with responses evoked by short MPIs displayed at the top. FIG. 10 lower panels, illustrate refractory recovery functions (round symbols) obtained from the waveforms in the upper panels. The fitted exponential decay function for each refractory recovery function (black line) and the resulting estimation for the refractory time (t0) are also provided. The participant and electrode number are included in the lower right corner of each panel. The stimulations were performed at the maximum comfortable level for each participant and electrode.

FIG. 11, upper panels, illustrate eCAP waveforms measured at different stimulation intensities for electrode 3 in one adult younger than 68 years (A08) and one adult older than 68 years (A54). Waveforms are arranged based on stimulation level (in current levels [CLs]), with responses evoked by the smallest stimulation level (i.e., eCAP threshold) displayed at the top. The largest stimulation level presented was the maximum comfortable level (i.e., C level). FIG. 11, lower panels, illustrate eCAP amplitude growth functions (AGFs, round symbols) obtained from the waveforms in the upper panels after converting stimulation levels to logarithmic units (dB re 1 nanocoulomb [nC]). The slope is provided for each eCAP AGF, along with a representation of the slope estimated with sliding window linear regression (solid black line). The participant and electrode number are included in the lower right corner of each panel.

FIG. 12 illustrates results of eCAP parameters (mean and standard deviation) measured for children with cochlear nerve deficiency (CND) and children with normal-sized cochlear nerves (NSCNs) used for model training. Ordered from left to right are the estimated absolute refractory recovery times (i.e., t0), eCAP thresholds, slopes of eCAP AGFs, and N1 peak latencies. Significant group differences are indicated with asterisks.

FIGS. 13A and 13B illustrate local electrode-neuron interface indices for children with normal-sized cochlear nerves and sensorineural hearing loss (S, FIG. 13A) and children with cochlear nerve deficiency (CND, FIG. 13B) included in the validation dataset.

FIG. 14 illustrates results of eCAP parameters (means and standard deviations) measured for adults younger than 68 years (black bars) and adults older than 68 years (white bars) at three electrode locations. Ordered in rows from top to bottom are the estimated absolute refractory recovery times (i.e., t0), eCAP thresholds, slopes of eCAP AGFs, and N1 peak latencies.

FIG. 15 illustrates the means and standard deviations of local electrode-neuron interface indices for adults younger than 68 years (black bars) and older than 68 years (white bars) at three electrode locations.

FIG. 16 illustrates local electrode-neuron interface indices for adult cochlear implant patients as a function of age at three electrode locations. Results from linear regression analyses are also shown.

FIG. 17 illustrates an exemplary computing device that can be used according to embodiments described herein.

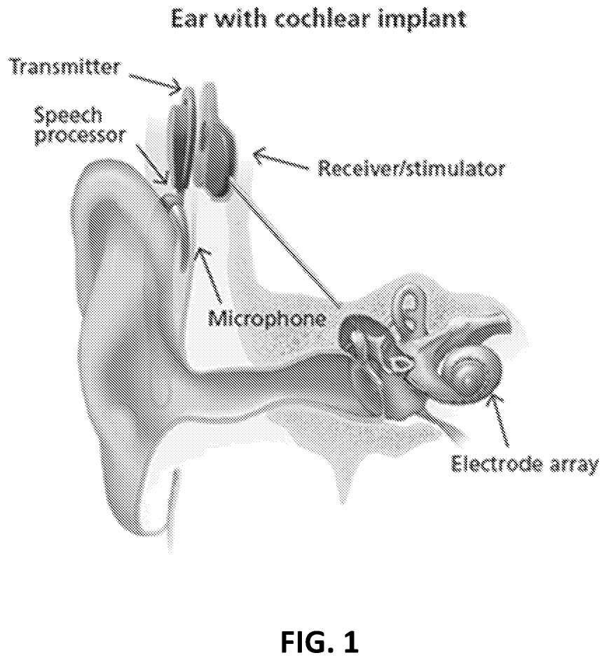

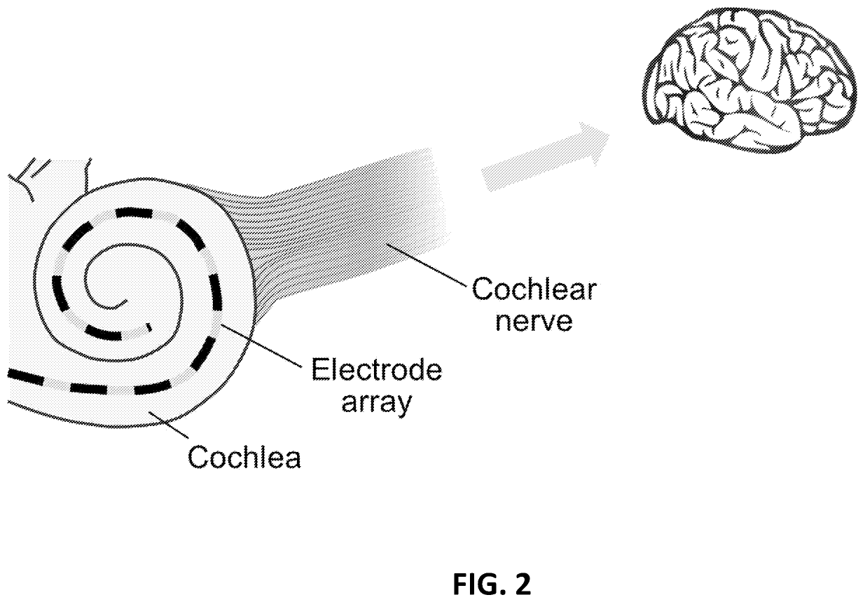

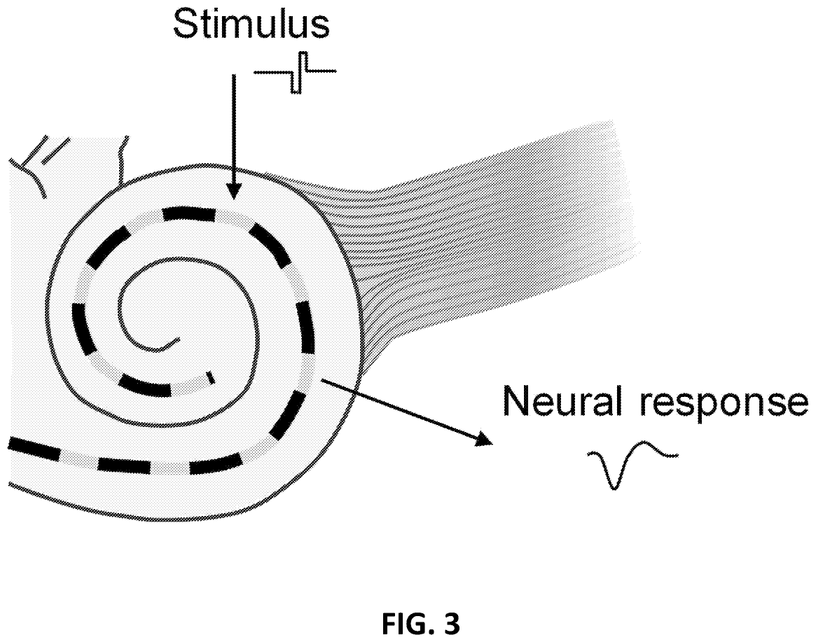

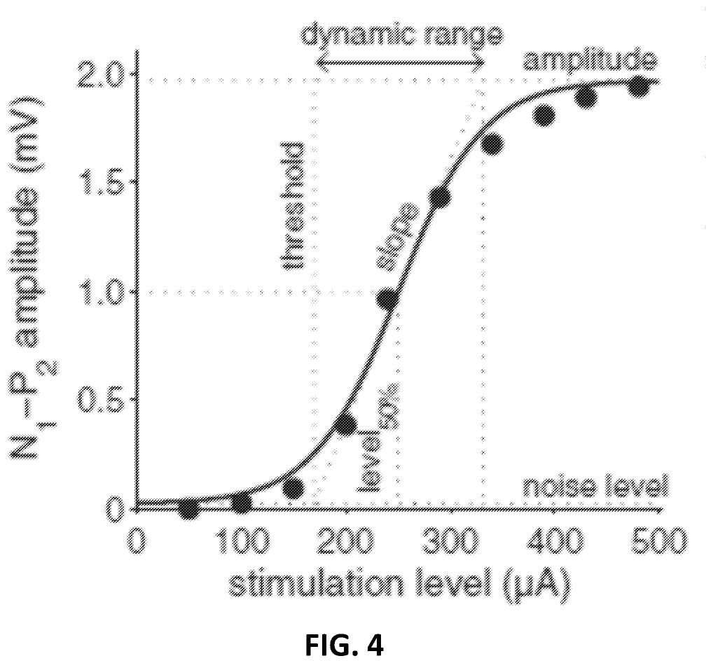

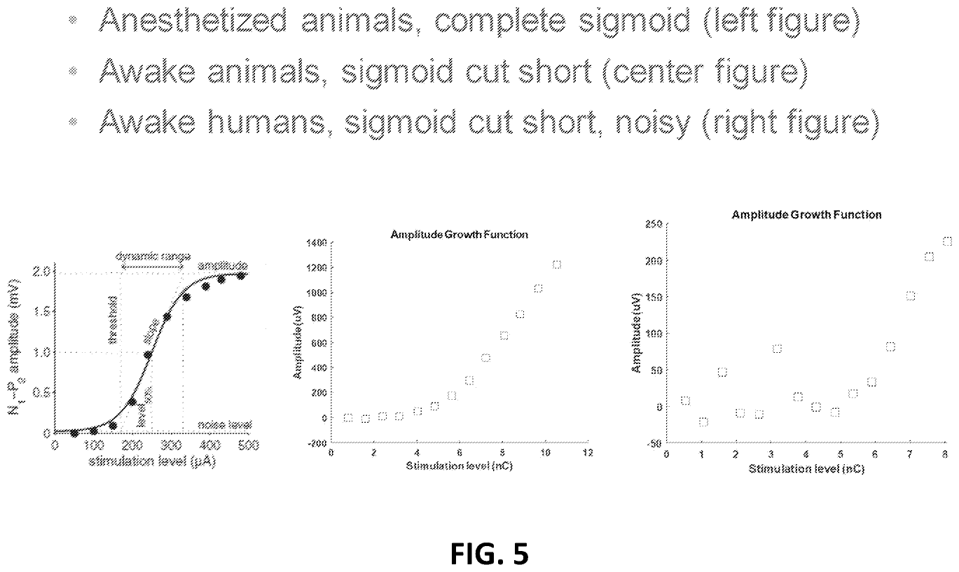

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present methods and systems may be understood more readily by reference to the following detailed description of preferred embodiments and the Examples included therein and to the Figures and their previous and following description.

### I. Detecting the Presence of eCAPS

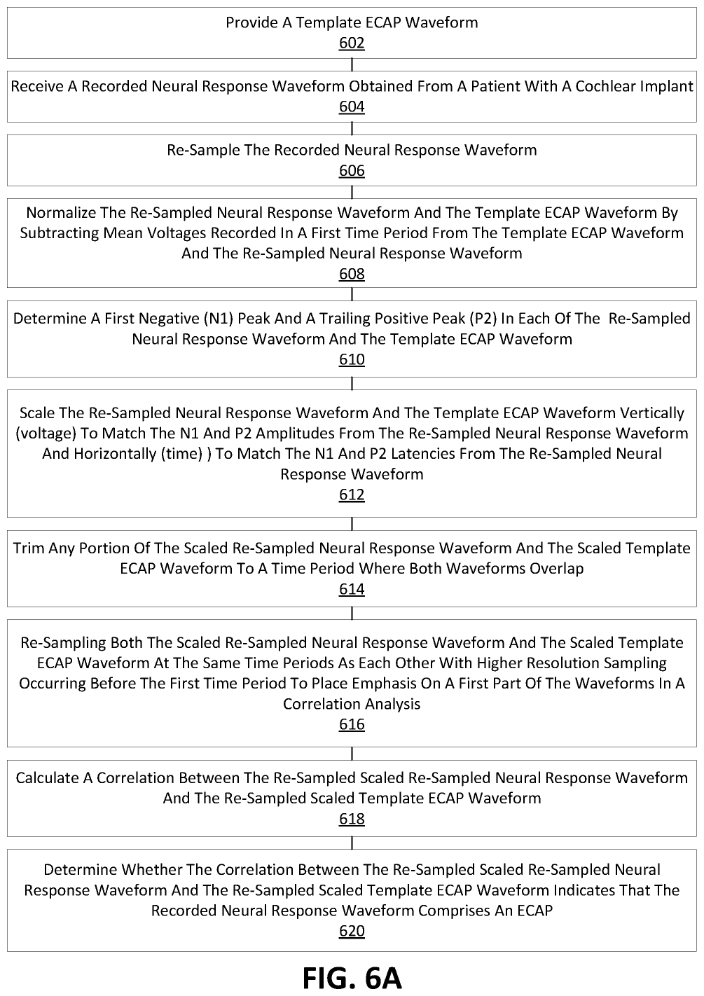

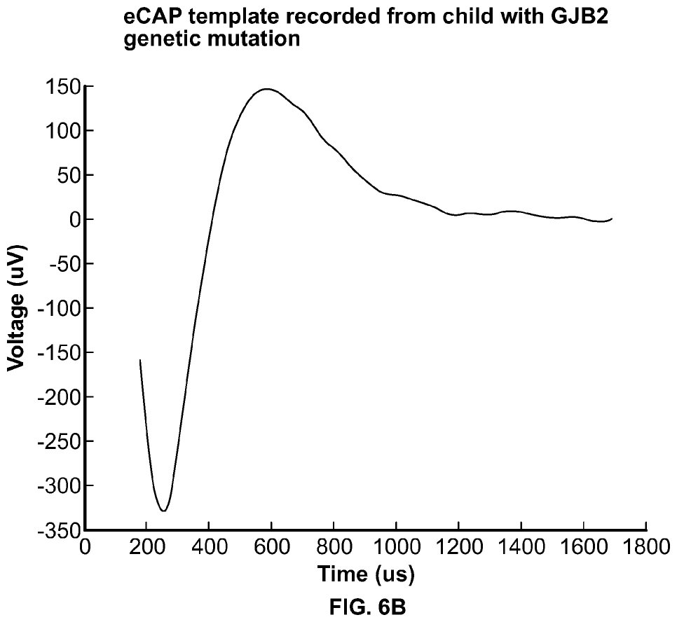

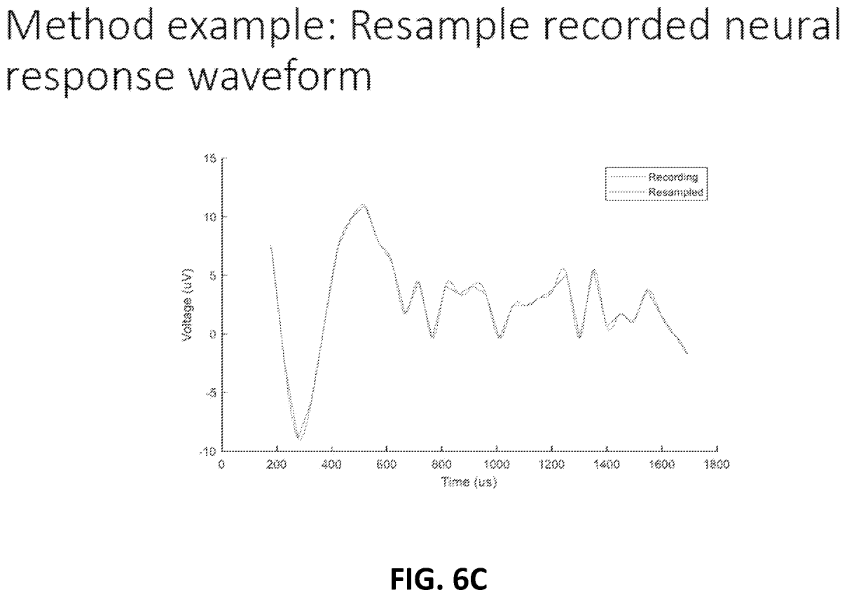

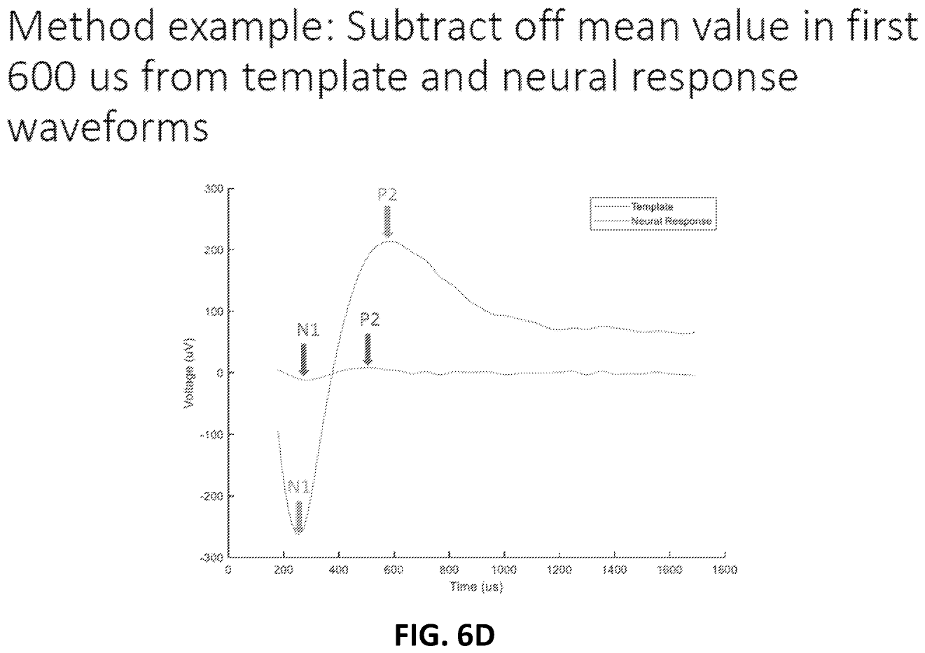

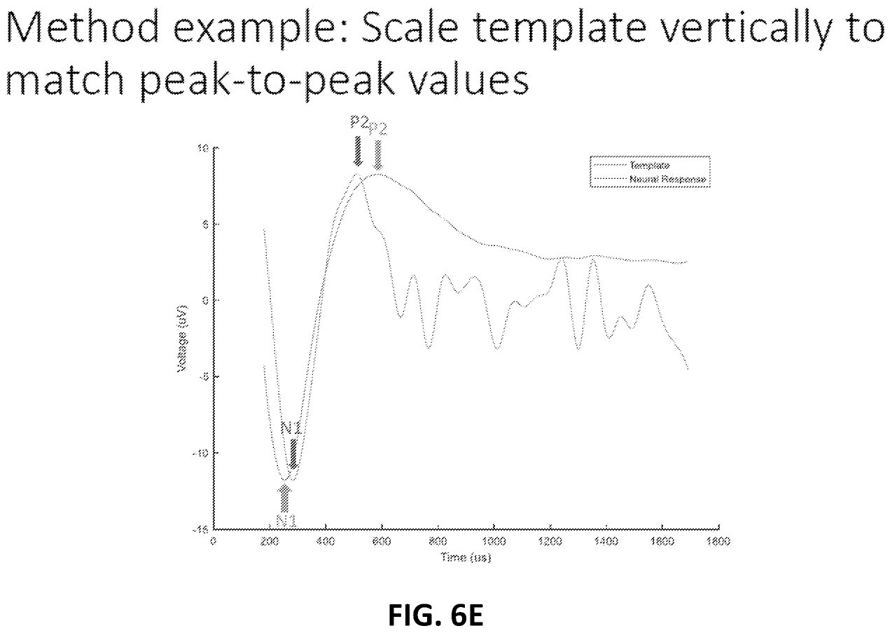

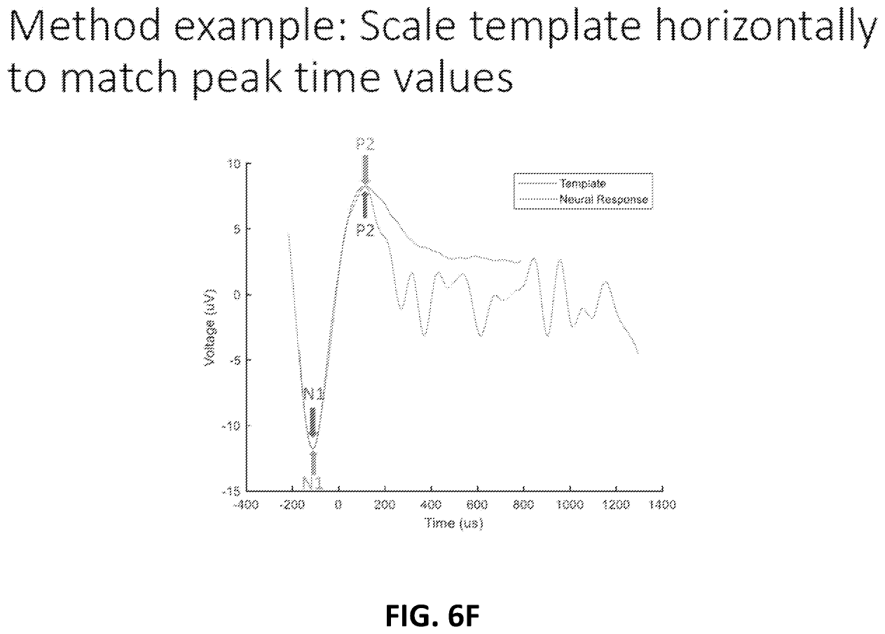

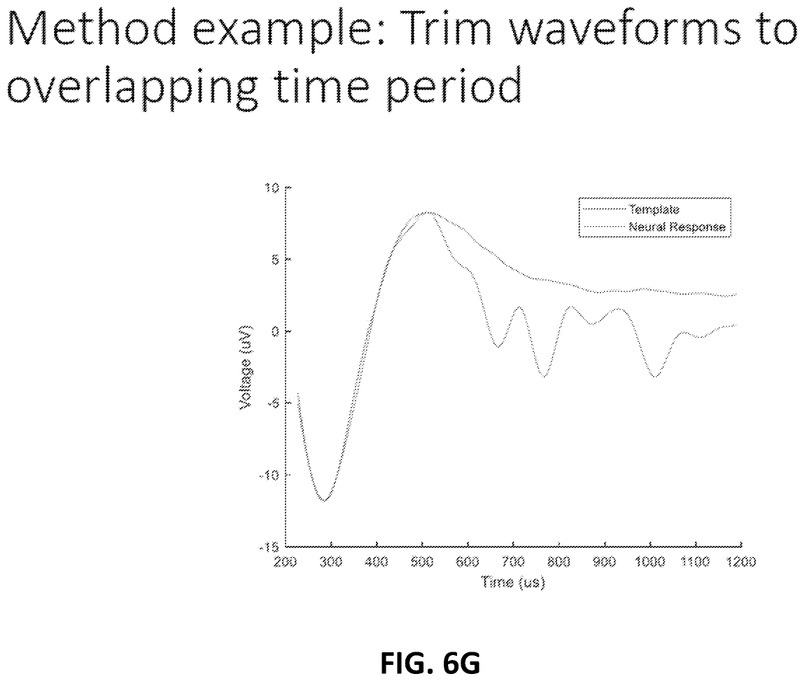

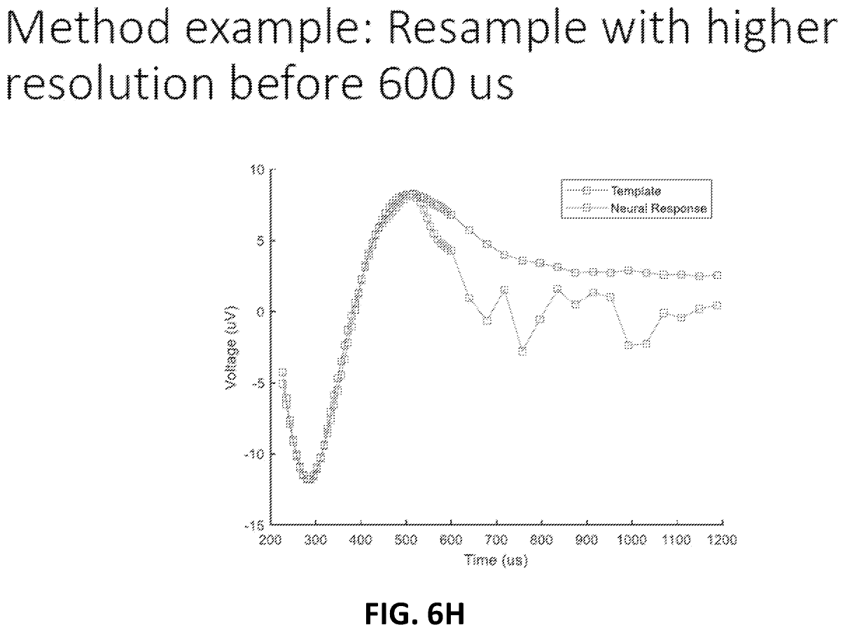

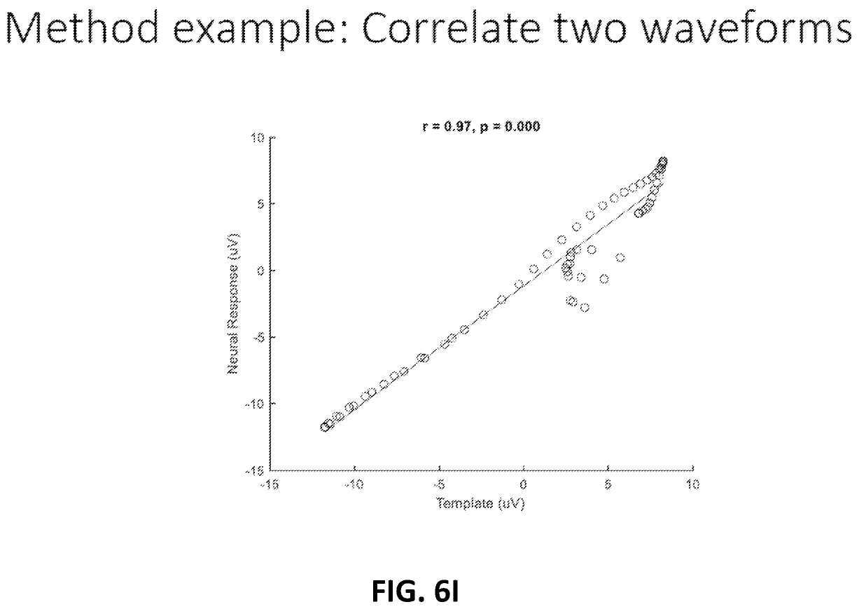

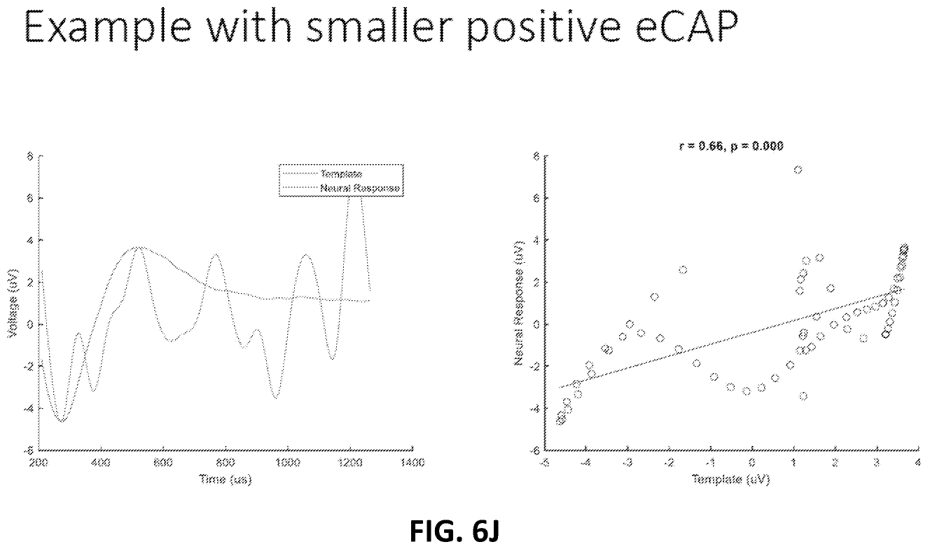

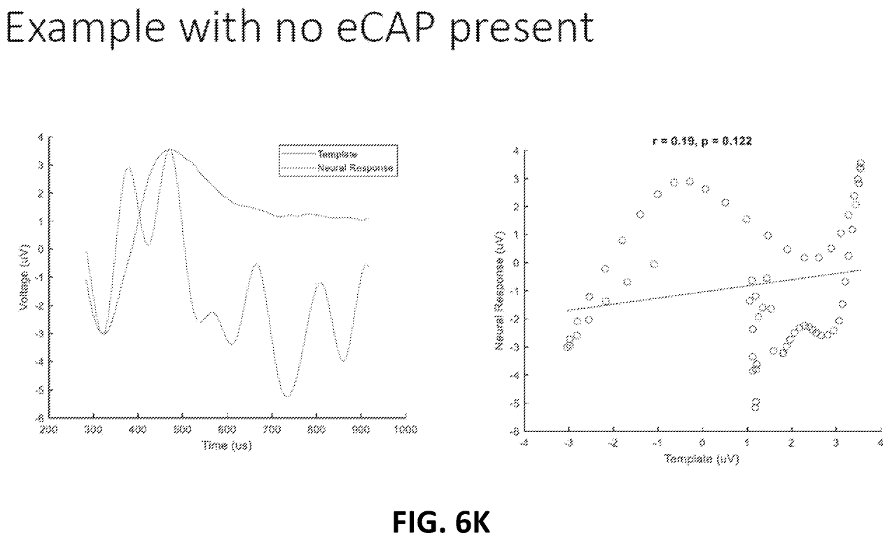

FIG. 6A illustrates an exemplary flowchart for a method of determining if an eCAP exists or not in a neural response. At 602, a template eCAP waveform is provided. FIG. 6B illustrates an example of a template eCAP waveform that was made from a neural response measured in a child with a GJB2 genetic mutation that causes hearing loss without impacting the auditory nerve. Therefore, the illustrated neural response is considered to be a template for a neural response. At 604, a recorded neural response obtained from a patient with a cochlear implant is received. As noted above, user defined stimuli can be sent through one electrode to stimulate the surrounding neurons. The neurons then respond and the electrical response can be recorded by a neighboring electrode, which comprises the neural response. At 606, the recorded neural response waveform is resampled. For example, the recorded neural response waveform may be up-sampled via cubic spline interpolation to create a smooth waveform at a higher effective sampling rate. FIG. 6C illustrates a comparison of the recorded neural response and the re-sampled neural response. At 608, the mean voltages recorded in the first 600 μ-sec are subtracted from the template and neural response waveforms. This is illustrated in FIG. 6D. At 610, the first negative (N1) peak and trailing positive peak (P2) are found in each waveform. This is also shown in FIG. 6D. At 612, the template waveform is scaled. As shown in FIG. 6E, the template waveform is scaled vertically (voltage) to match the N1 and P2 amplitudes from the neural response waveform. As shown in FIG. 6F, the template waveform is then scaled horizontally (time) to match the N1 and P2 latencies from the neural response waveform. At 614, the waveforms are then trimmed to the time period that overlaps after scaling. This is illustrated in FIG. 6G. At 616, the waveforms are resampled at the same time periods as each other with higher resolution sampling occurring before 600 μ-sec to place emphasis on the first part of the waveforms in the correlation analysis. This is illustrated in FIG. 6H. At 618, a correlation between the template and neural response waveforms is calculated. For example, the Pearson correlation between the template and neural response waveform may be calculated. This is illustrated in FIG. 6I. At 620, it is determined whether the correlation between the neural response and the eCAP template indicates that the neural response comprises an eCAP. For example, if the correlation is the Pearson correlation and the correlation value is larger than 0.6 and the estimated eCAP amplitude is greater than 5 uV (noise floor of the device), an eCAP is determined to be present. Otherwise, an eCAP is determined to not be present. FIG. 6J illustrates an example correlation where the neural response is an eCAP (correlation value greater than 0.6), and FIG. 6K illustrates an example correlation where the neural response is not an eCAP (less than 0.6 correlation value). It is to be appreciated that different methods of determining the correlation between the eCAP template and the neural response are contemplated within the scope of this invention as well as differing correlation values for deciding whether a neural response comprises an eCAP, or not.

In various implementations, the above-described methods determine whether a neural response comprises an eCAP. In some instances implementations of the method may be used in post-surgery software package for clinicians to optimize and personalize the cochlear implant settings for individual patients. The specific settings that could be informed include “Enable/disable an electrode” and “pulse phase duration.” Furthermore, implementations of the method could be included in clinical and/or research software platforms as the disclosed technology reduces the advanced training that an audiologist would need to apply objective measures into clinical practice.

### II. Estimating Survival of Auditory Nerve Fibers

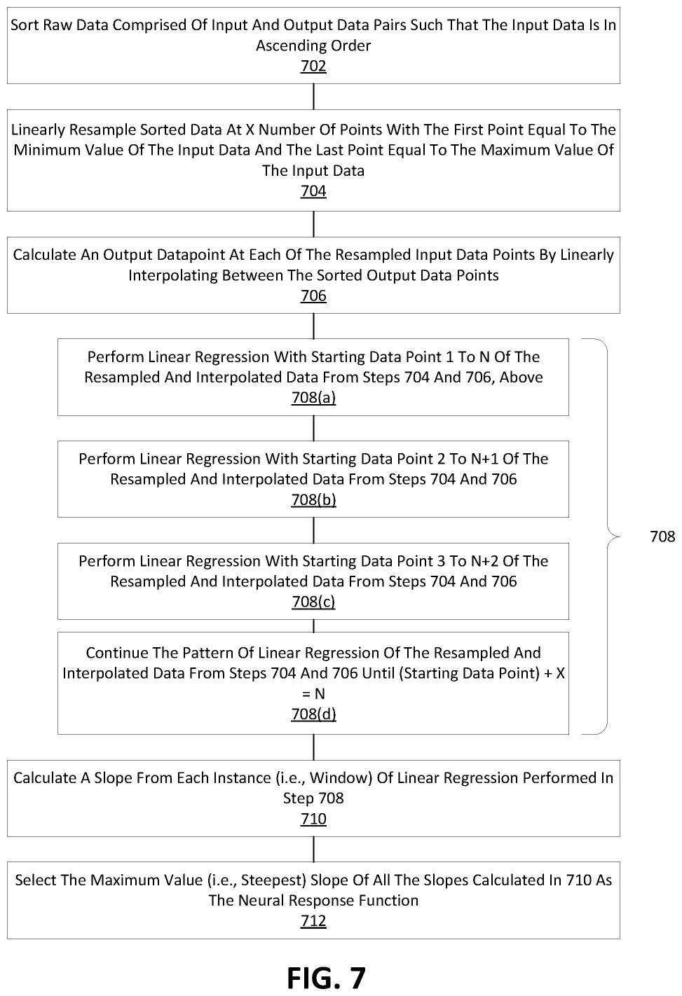

FIG. 7 illustrates an exemplary flowchart for a method of providing an estimate for the slope of a neural response function characterized by a series of stimulation levels (i.e. input data) and corresponding amplitude of neural responses (i.e. output data). These functions are often called amplitude growth functions (AGFs) or input/output (I/O) functions. For example, the raw data comprises x,y pairs of stimulation level (input data) and corresponding eCAP amplitudes (output data). The stimulation level can be specified in any units. Microamps or nanocoulombs are typical. The amplitudes of neural responses can be specified in any units. Microvolts is typical.

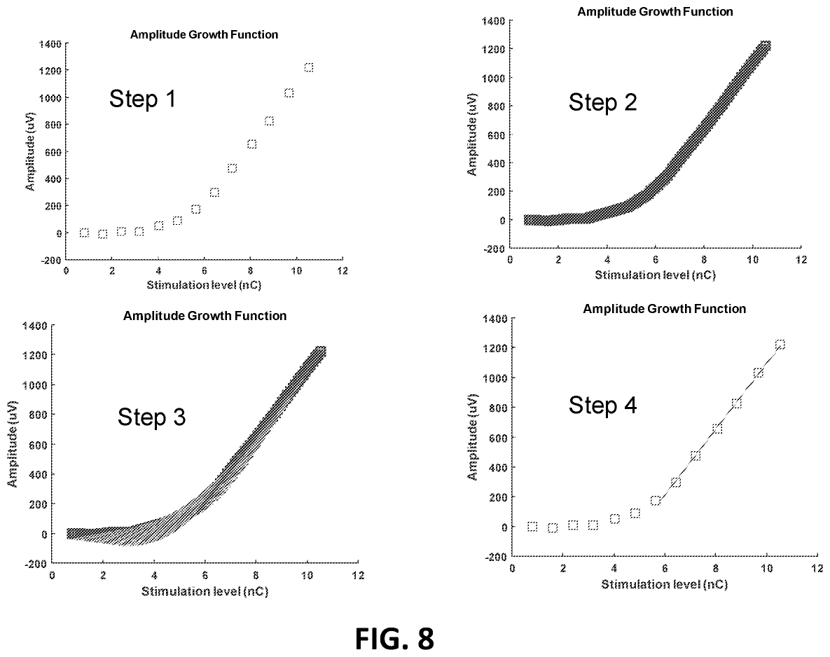

One embodiment of the method comprises 702, sorting raw data comprised of input and output data pairs such that the input data is in ascending order. At 704, the sorted input data from 702 is linearly resampled at X number of points with the first point equal to the minimum value of the input data and the last point equal to the maximum value of the input data. X can be any positive integer equal to or greater than the number of data points in the input data. A typical value for X is 100. This is illustrated in the panel labeled “Step 1” of FIG. 8. At 706, an output datapoint is calculated at each of the resampled input data points from 704 by linearly interpolating between the sorted output data points. This is illustrated in the panel labeled “Step 2” of FIG. 8. At 708, linear regression is performed on a moving window of N number of data points. N is any positive integer less than or equal to X. For example, at 708(a) linear regression is performed with starting data point 1 to N of the resampled and interpolated data from steps 704 and 706. At 708(b), linear regression is performed with starting data point 2 to N+1 of the resampled and interpolated data from steps 704 and 706. At 708(c), linear regression is performed with starting data point 3 to N+2 of the resampled and interpolated data from steps. At 708(d), this pattern of linear regression continues until (starting data point)+N=X. At 710, a slope is calculated from each instance (i.e., window) of linear regression in step 708. This is illustrated in the panel labeled “Step 3” of FIG. 8. Slope is defined as m in the linear regression equation y=mx+b. At 712, the maximum value (i.e., steepest) of all the slopes calculated in step 710 is selected as the slope of the neural response function. This is illustrated in the panel labeled “Step 4” of FIG. 8.

In some instances, the method of FIG. 7 comprises an adaptive and iterative algorithm that chooses the number of points (X and N) depending on the input and output data. It is also to be noted that a steeper slope correlates with a greater number of neuron survival in the cochlear nerve (i.e., greater values of slope indicate greater neural survival and/or function).

### A. Examples of Estimating Survival of Auditory Nerve Fibers

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process.

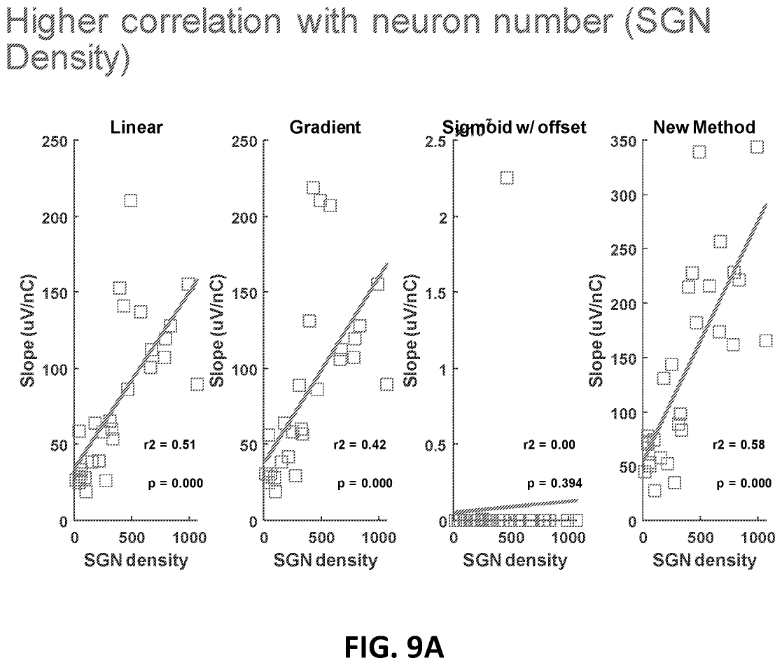

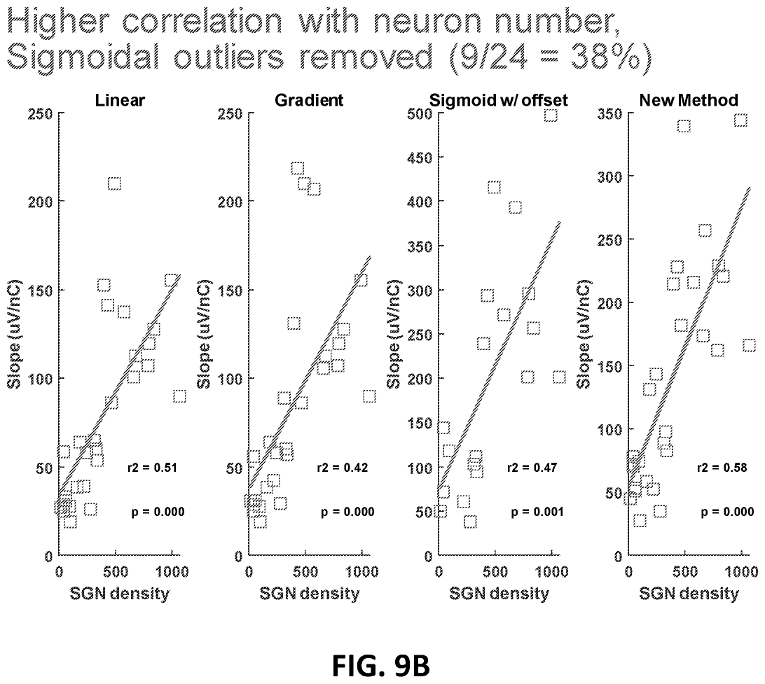

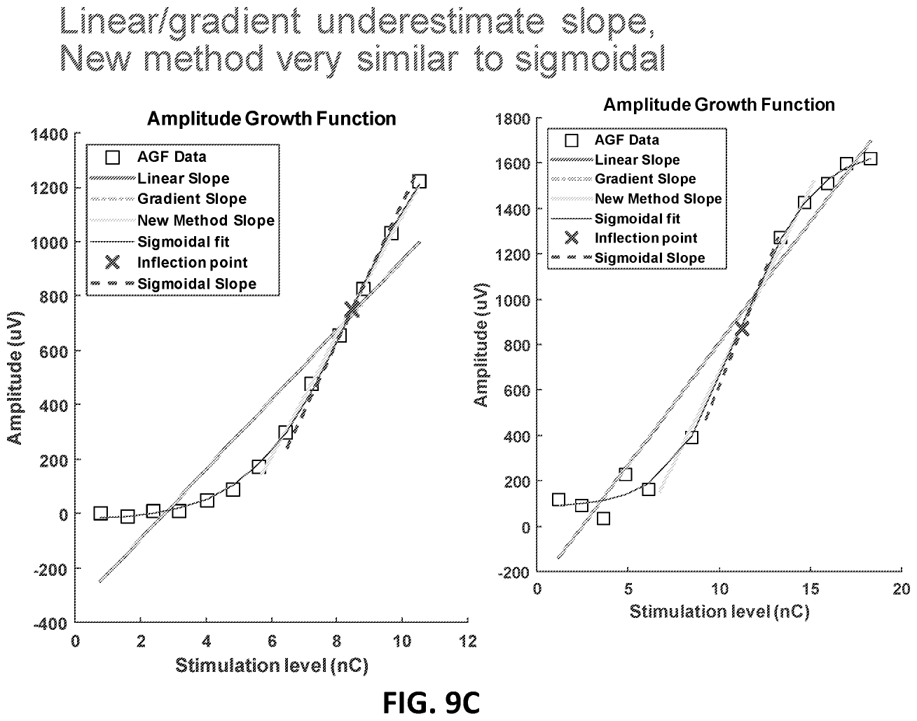

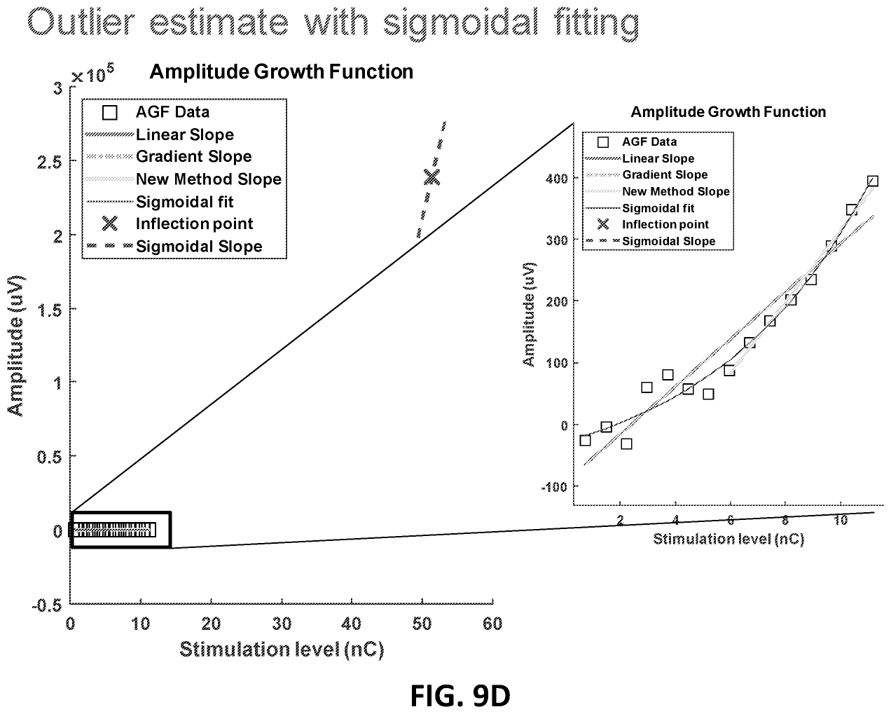

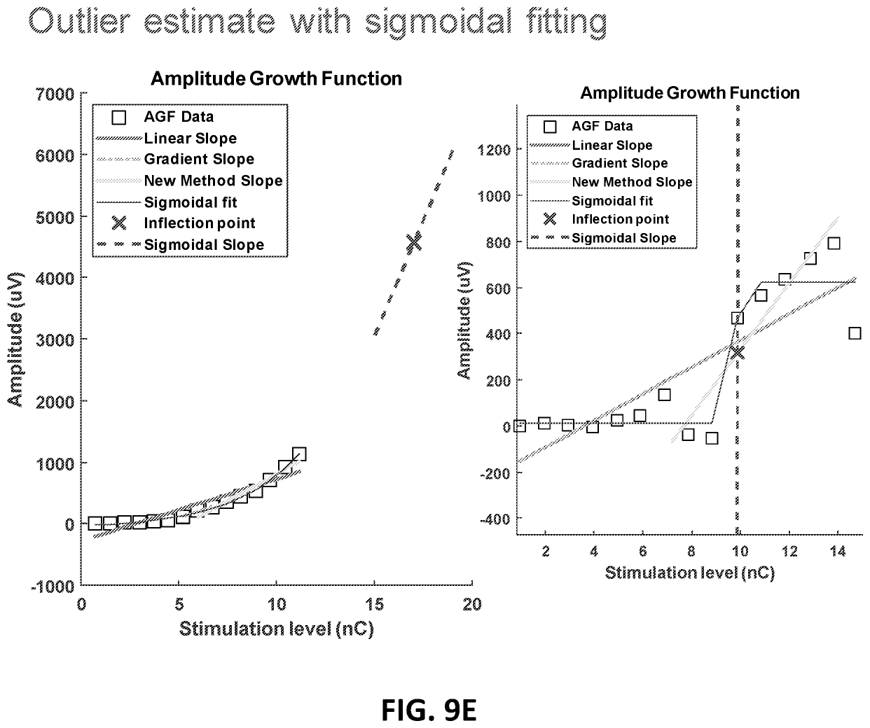

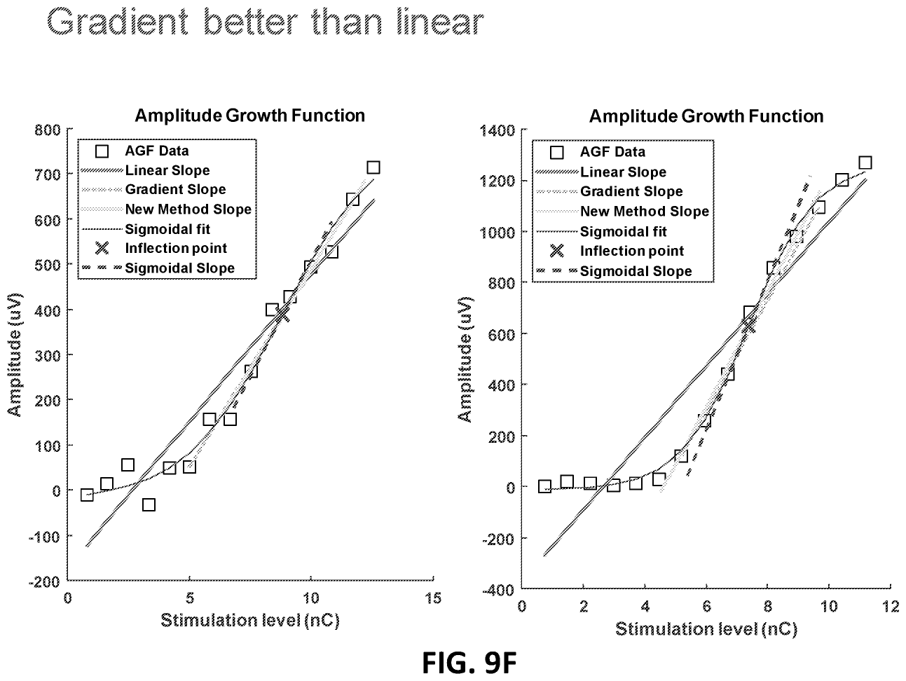

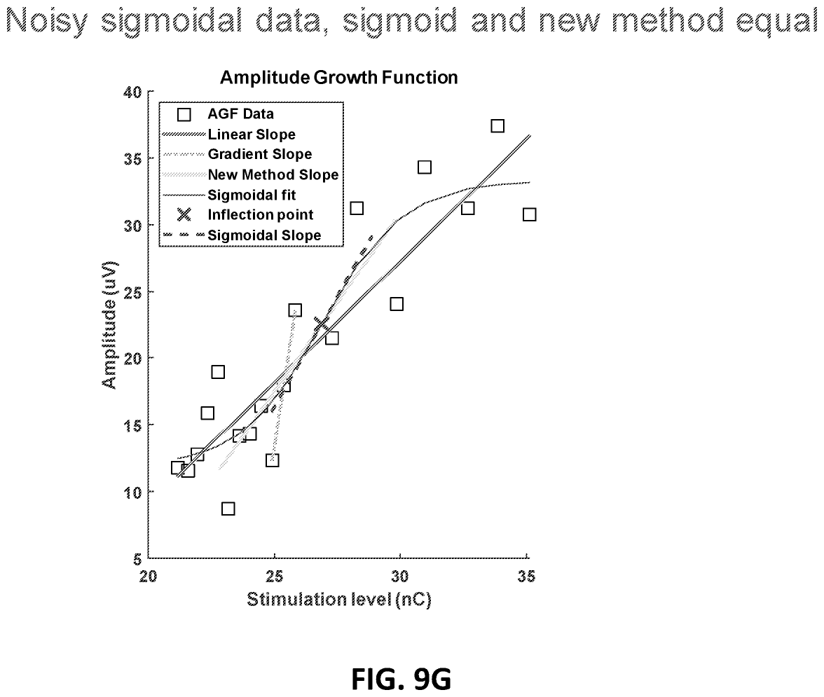

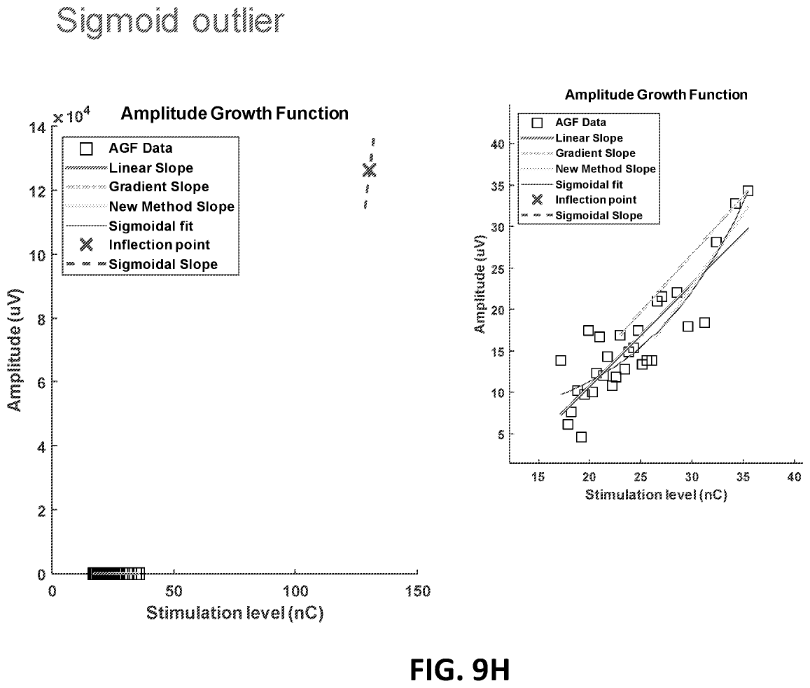

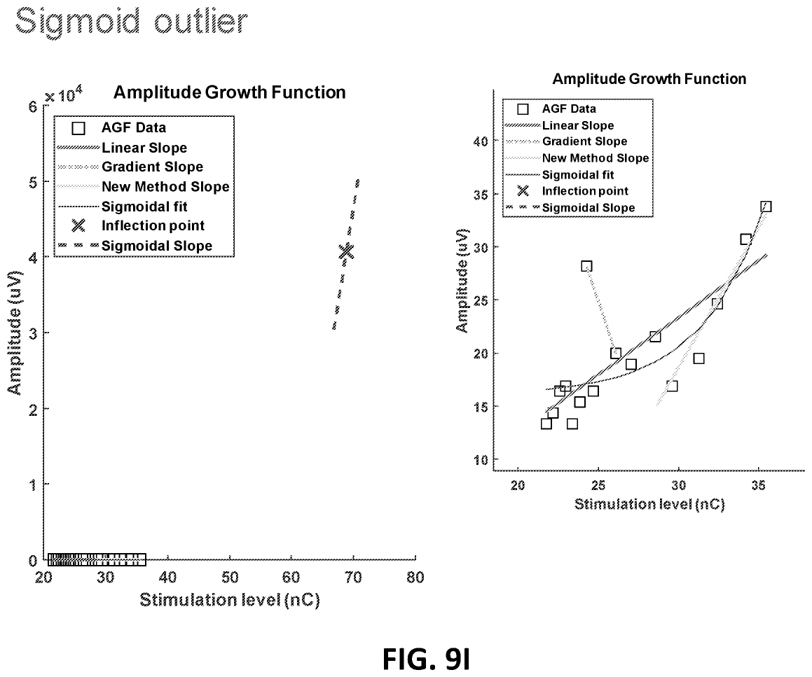

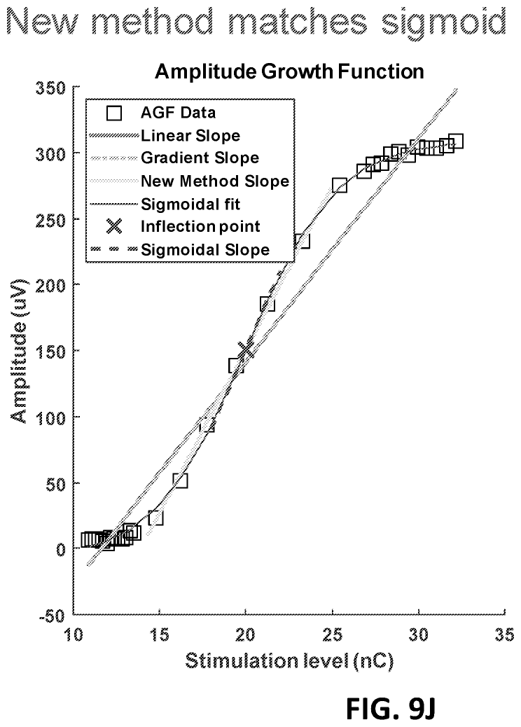

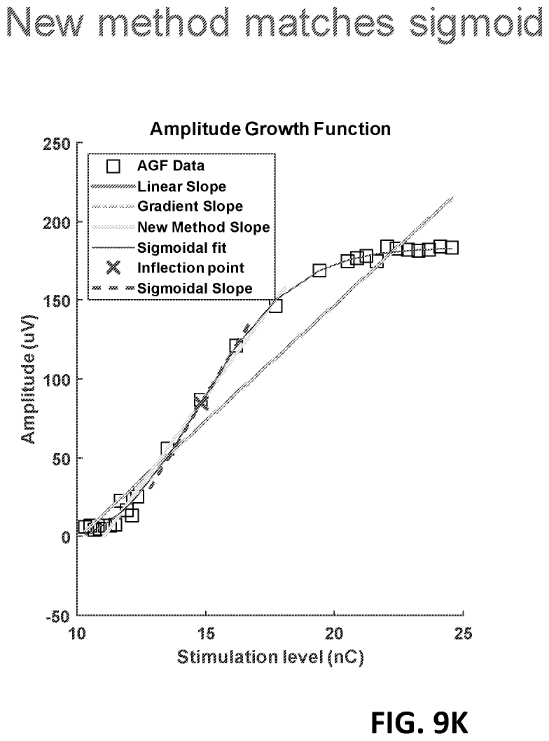

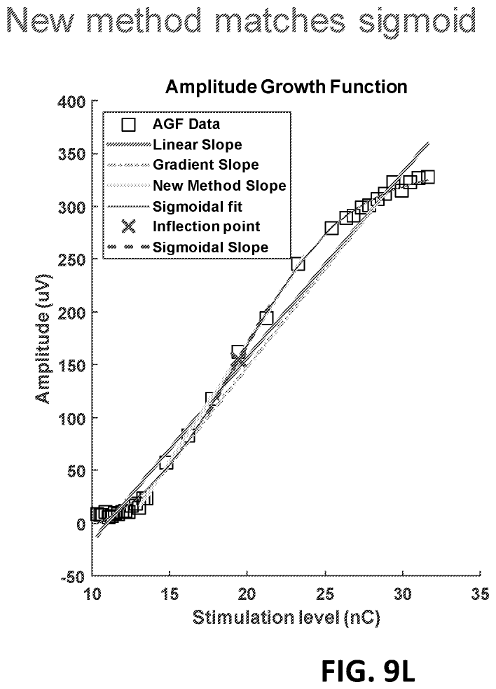

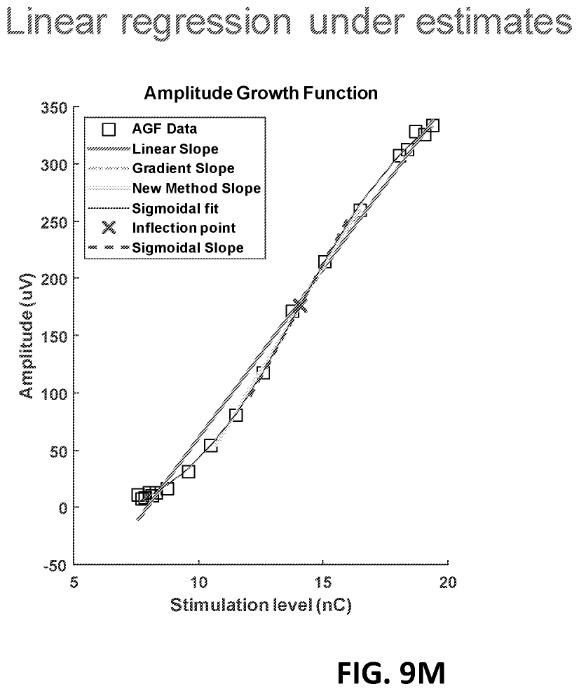

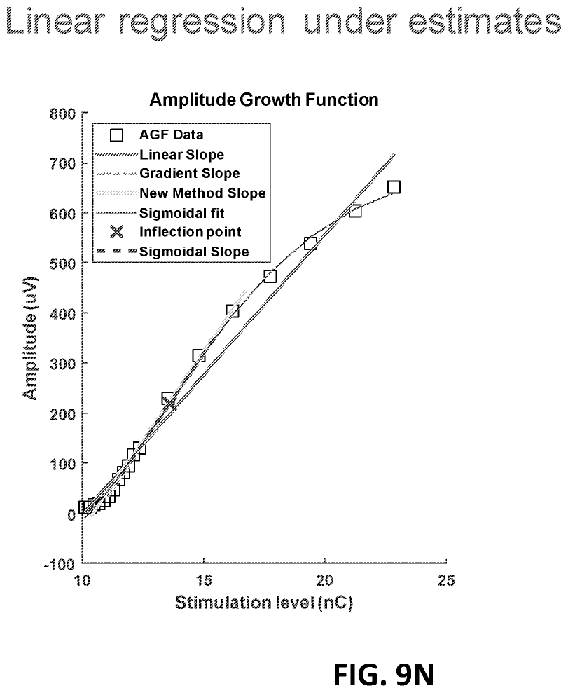

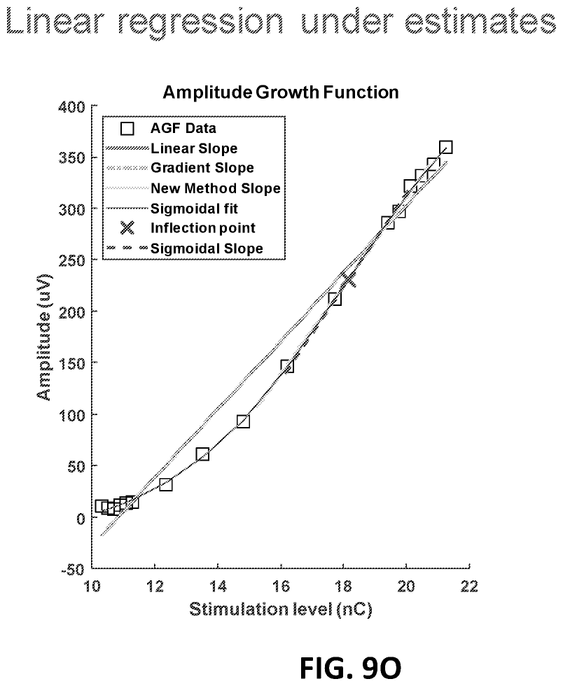

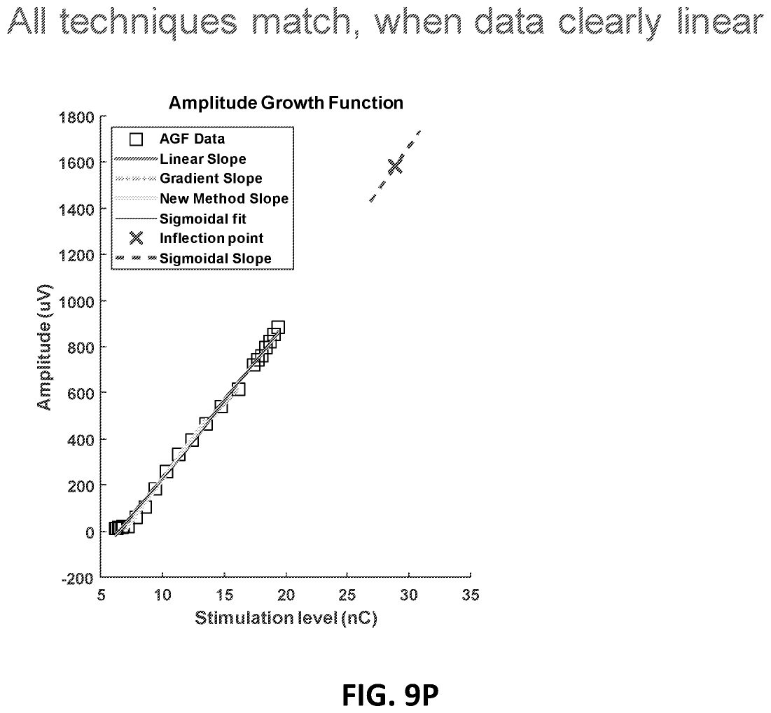

FIGS. 9A-9P illustrate comparisons of calculating the slope of the AGF data using the disclosed embodiments and conventional methods of calculating the slope, in both animals and humans. FIGS. 9A-9F are based on animal data. FIGS. 9G-9P are based on human data.

### B. Appendix to Estimating Survival of Auditory Nerve Fibers

Attached hereto and made a part hereof is Appendix A, “Prediction of the functional status of the cochlear nerve in individual cochlear implant users using machine learning and electrophysiological measures,” which is fully incorporated by reference. This Appendix A describes development of a cochlear nerve index in which the slope of the sigmoid curves (i.e. eCAP AGFs), as determined by the systems and methods described herein, is used as a factor in determination of the cochlear nerve index.

### C. Conclusion to Estimating Survival of Auditory Nerve Fibers

Advantages of the disclosed embodiments include 1) it works for all AGFs, which means that (a) It finds the same slope as the sigmoidal slope for animal and human AGFs that reach saturation; (b) it finds the linear portion of the AGF for AGF's that don't reach saturation; it works regardless of the number of data points in the AGF, including data points that are uniformly or non-uniformly distributed. In human patients, these are usually non-uniformly distributed which causes issues for other methods of calculating the slope of the raw data; (c) it works with noisy, raw (i.e. no preprocessing) AGFs. For example, in one exemplary test, the disclosed method gives very similar slope estimations for 28 animals whether or not the data points less than 100 uV (i.e., excluding outliers) are included or not; (d) it does not require user input. This is related to (c). For example, a threshold of 100 uV to exclude data does not need to be provided. Given the thousands of human AGFs that are possible, it would be impossible to find a suitable threshold below which to exclude the nonlinear portion of the AGF. Therefore, this feature is especially advantageous from a clinical perspective, in which a slope estimation could be provided without the clinician making any decision on how to process the AGF (i.e. include/exclude data points); (e) it does not create unrealistic estimations of the slope. This is difficult to quantify, but there were no unrealistic, extreme values for the 28 AGFs from guinea pigs, 317 AGFs from adults, and 963 AGFs from children that were tested using the disclosed embodiments. In contrast, the sigmoidal slope with offset had 33% unrealistic estimates (slope >2000 uV/nC) for the guinea pig data. It was about 9% of the adult AGFs and 15% of the children AGFs.

Applications for the disclosed embodiments include clinical care for cochlear implant patients. Specifically, this includes 1) optimizing fine tuning of cochlear implant programming settings for individual patients, and 2) improving proprietary internal programming settings of cochlear implants

In current clinical practice, patients receive a cochlear implant with default settings. There is a very wide variety of speech recognition performance among cochlear implant patients. Some patients do really well with the default settings, some patients don't do well at all. After surgery, these parameters can be adjusted by the clinician by “trial and error.” The clinician will modify some parameters and the patient will report back if they think they are able to hear better or worse. This current practice is inefficient, time-consuming, frustrating, and subjective. The disclosed embodiments allow clinicians to objectively estimate the function of the patient's nerves near each electrode location. Based on these results, they can make informed decisions on how to modify the programming settings. Below is an outline how a clinical visit would be conducted using the disclosed embodiments:
a. The hardware and software from the manufacturer of the device that the patient has implanted is used to collect a series of measurements to create an amplitude growth function (AGF). This is a standard feature of all manufacturers' available software and hardware.
b. The slope of the eCAP AGF is calculated using the disclosed embodiments. This could be implemented either (i) incorporated as a part of the manufacturer's software; (ii) as a stand-alone-software package. Each manufacturer allows the raw datapoints to be exported. So they could be imported into a separate software package to calculate the slope.
c. The slope of the eCAP AGF provides the clinician an estimate of how well the nerves function for the specific patient and can adjust the programming settings accordingly. These adjustments can include deactivating electrodes of the cochlear implant near nerves that do not function properly and/or passing more stimulation/information through regions with the best functioning nerves.
d. Repeat a-c for each electrode in the cochlear implant; resulting in
e. Optimized settings of the cochlear implant that help the patient hear better,

Generally, the internal workings of cochlear implants is proprietary and a “black box.” However, in some instances outputs of the disclosed embodiments can be used as feedback to the cochlear implant to dynamically “tune” it according to a patient's specific neural responses.

### III. Use of eCAP: Determining Effects of Advanced Age on the Electrode-Neuron Interface

A study was conducted with participants included 30 post-lingually deafened (i.e., lost hearing after twelve years of age) adult CI users. All participants were implanted with a Cochlear™ 135 Nucleus® device (Cochlear Ltd., Sydney, NSW, Australia) with a full electrode insertion in the test ear. Twenty participants were implanted unilaterally and ten participants (A03, 137 A07, A08, A11, A19, A27, A34, A48, A49, and A50) were implanted bilaterally. For all participants, only one ear was tested for this study. For the bilateral CI users, the test ear was selected pseudo-randomly using a pseudo-random number generator.

The participants were separated by age at testing into two study groups with 15 participants in each group: younger (age ≤68 years) and older (age >68 years). For the younger study group, the age at testing ranged from 48.4 to 67.6 years (mean: 60.3 years, SD: 5.9 years). For the older study group, the age at testing ranged from 69.0 to 83.2 years (mean: 75.8 years, SD: 4.6 years). Detailed demographic information of the study 145 participants is provided in Table 1, below.

TABLE 1

| | | | | | | | |
|---|---|---|---|---|---|---|---|
| Subject | | Ear | AAI | AAT | Internal Device and | Etiology of | Electrodes |
| Number | Sex | Tested | (yrs) | (yrs) | Electrode Array | Hearing Less | Tested |

Younger:

| | | | | | | | |
|---|---|---|---|---|---|---|---|
| A03 | M | L | 58.8 | 61.8 | CI512 | SHL | 3, 12, 21 |
| A07 | M | R | 43.3 | 52.7 | 24RE (CA) | Unknown | 3, 12, 21 |
| A08 | F | R | 54.4 | 67.6 | 24RE (CA) | Hereditary | 3, 12, 21 |
| A17 | M | R | 56.2 | 62.5 | 24RE | Unknown | 6, 12, 21 |
| A19 | F | L | 54.6 | 55.4 | CI532 | Rubella | 4, 12, 21 |
| A20 | M | R | 60.3 | 62.7 | CI522 | Trauma | 6, 12, 21 |
| A27 | F | R | 44.5 | 48.4 | CI422 | Unknown | 3, 12, 21 |
| A29 | F | R | 48.5 | 59.6 | 24RE (CA) | Hereditary | 3, 12, 21 |
| A30 | F | R | 64.9 | 65.6 | CI532 | Unknown | 3, 12, 21 |
| A40 | M | R | 59.0 | 59.5 | CI532 | Unknown | 3, 12, 21 |
| A47 | F | R | 59.1 | 66.9 | 24RE (CA) | Unknown | 3, 12, 21 |
| A48 | F | L | 57.3 | 59.7 | CI532 | Hereditary | 3, 12, 21 |
| A49 | M | L | 51.5 | 52.7 | CI532 | Unknown | 3, 12, 21 |
| A51 | M | R | 55.8 | 62.4 | 24RE (CA) | Unknown | 3, 12, 21 |
| A52 | M | L | 65.2 | 67.6 | CI532 | Tumor | 3, 12, 21 |

Older:

| | | | | | | | |
|---|---|---|---|---|---|---|---|
| A06 | M | L | 60.7 | 69.0 | CI512 | Meniere's | 3, 12, 18 |
| A11 | M | R | 77.5 | 80.7 | 24RE (CA) | Noise | 3, 12, 21 |
| A25 | M | L | 74.9 | 83.2 | 24RE (CA) | Unknown | 3, 12, 21 |
| A34 | M | R | 68.7 | 70.4 | CI532 | Trauma | 3, 12, 21 |
| A35 | F | L | 72.4 | 76.6 | CI422 | Noise | 3, 12, 21 |
| A38 | M | L | 74.0 | 79.0 | CI422 | Ototoxicity | 3, 12, 21 |
| A41 | F | R | 73.3 | 79.8 | 24RE (CA) | Hereditary | 3, 12, 21 |
| A44 | M | L | 68.4 | 70.2 | CI532 | Noise | 3, 11, 20 |
| A45 | F | L | 67.5 | 79.4 | 24RE (CA) | Autoimmune | 3, 12, 21 |
| A46 | M | R | 76.2 | 78.3 | CI532 | Noise | 3, 12, 21 |
| A50 | M | R | 75.2 | 76.7 | CI522 | Noise | 3, 12, 21 |
| A54 | M | L | 69.3 | 69.7 | CI622 | Noise | 3, 12, 21 |
| A55 | M | R | 68.2 | 70.9 | CI532 | Unknown | 3, 12, 21 |
| A56 | F | L | 65.9 | 77.3 | 24RE (CA) | Noise | 6, 12, 21 |
| A57 | M | R | 73.7 | 76.5 | CI532 | Unknown | 3, 12, 21 |

Demographic information of all subjects who participated in this study, listed by study group. Definitions: AAT, age at implantation; AAT, age at testing; 24RE (CA): Freedom Contour Advance electrode array; SHL, sudden hearing loss

All participants were tested for eCAP measures at three electrode locations across the electrode array, typically electrodes 3, 12, and 21 (see Table 1). All participants had a full electrode array insertion with a Cochlear® Nucleus™ CI (Cochlear Ltd., Sydney, NSW, Australia), which means that electrodes 1 and 22 were placed near the base and the apex of the cochlea, respectively. Therefore, the three testing electrodes were referred to as the “basal”, the “middle” and the “apical” electrode based on their relative locations along the electrode array.

The procedures for obtaining the eCAP in adult CI users were the same as those used in a previous study (Skidmore et al., 2021a—see Appendix A, attached hereto and made a part hereof). Briefly, eCAP measures were acquired using the Advanced Neural Response Telemetry function via the Custom Sound EP (v. 4.3 or 5.1) software interface (Cochlear Ltd, Sydney, NSW, Australia). The stimulus was a symmetric, cathodic-leading, biphasic pulse with an interphase gap of 7 μs and a pulse phase duration of 25 μs/phase. Other recording parameters included a 15 Hz probe rate, an amplifier gain of 50 dB, sampling delays between 98 and 122 μs, an effective sampling rate of 20 kHz, and 50 sweeps per averaged eCAP response. The stimulus was presented to individual CI electrodes in a monopolar-coupled configuration via a N6 sound processor that was connected to a programming pod.

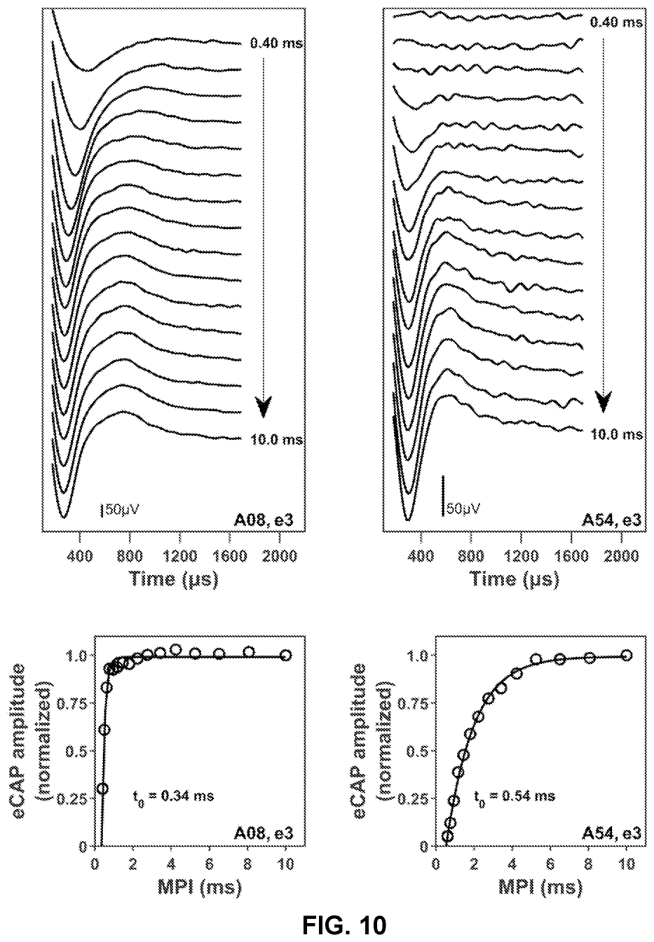

The eCAP refractory recovery function (RRF) was obtained with two electrical pulses (marker pulse and probe pulse) using a modified template subtraction method (Miller et al., 2000). The masker pulse was presented at the participants' maximum comfortable level (i.e., C level), and the probe pulse was presented at 10 current levels (CLs) below C level. A series of eCAPs were recorded as the MPI was systematically increased from 400 μs to 10 ms. The top panels of FIG. 10 show traces recorded at different masker-probe intervals (MPIs) for electrical stimulations at electrode 3 in one younger adult (A08) and one older adult (A54).

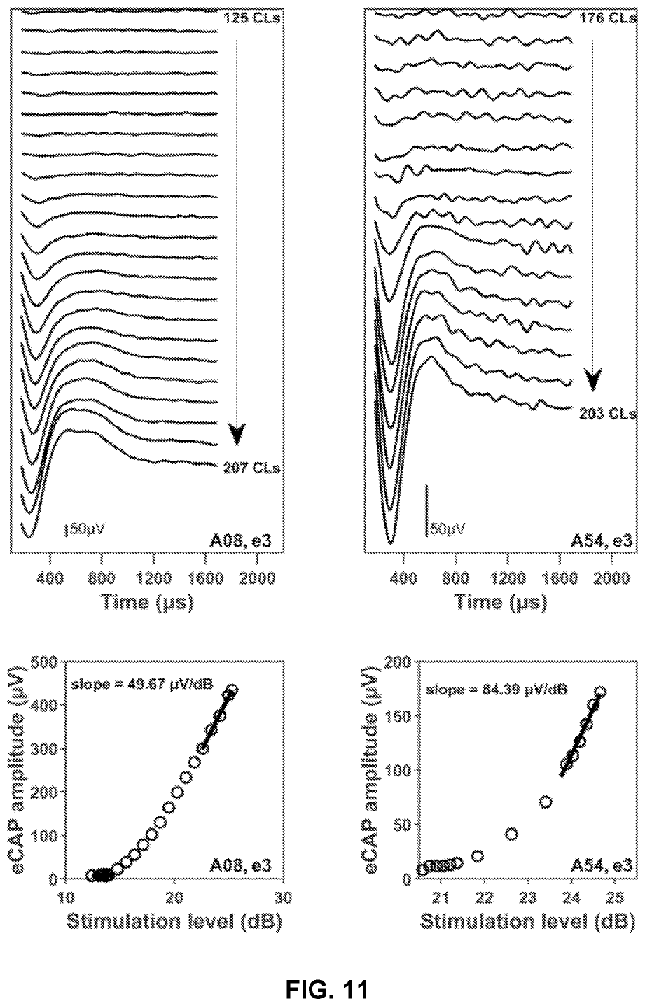

The eCAP AGF was obtained using the forward-masking-paradigm (Brown et al., 1990). The masker pulse, which was always presented at 10 CLs higher than the probe pulse, was initially presented at C level and decreased by 1 CL for at least five measurements. This was followed by a systematic decrease in steps of 5 CLs until no eCAP response could be visually identified. The stimulation level was then subsequently increased in steps of 1 CL until at least five eCAPs were measured using this small step size. The top panels of FIG. 11 show eCAPs recorded at different stimulation levels at electrode 3 in one younger adult (A08) and one older adult (A54).

### A. Data Analysis

The eCAP amplitudes measured at different MPIs were normalized to the eCAP amplitude measured at 10 ms and plotted as a function of MPI to generate the eCAP RRF. An estimate of the absolute refractory period (i.e., t₀) was found using statistical modeling with the exponential decay function

$${eCAP}_{N} = A ( 1 - {e}^{- ( \frac{MPI - {t}_{0}}{τ} )} ) \qquad ( 1 )$$

where eCAP_{N} is the normalized eCAP amplitude, A represents the maximum normalized eCAP amplitude, MPI is the masker probe interval in ms, and τ is an estimate of the relative refractory period. The absolute refractory period has been estimated using this equation in several previously published studies (e.g., Morsnowski et al., 2006; Botros & 199 Psarros, 2010; Wiemes et al., 2016; He et al., 2018). The bottom panels of FIG. 10 show the eCAP RRFs obtained from the eCAP recordings shown in the top panels, along with the fitted exponential decay functions used to estimate the absolute refractory period. No eCAPs were visually identified in the traces recorded for participant A54 at MPIs of 400 and 496 μs and so they were excluded from the eCAP RRF.

The eCAP AGF was generated by plotting the eCAP amplitudes (in μV) as a function of the corresponding stimulation level (in dB re 1 nanocoulomb [nC]). The bottom panels of FIG. 11 show the eCAP AGFs obtained from the eCAP recordings shown in the top panels, along with an estimate of the maximum slope of the eCAP AGF.

For each eCAP AGF, the maximum slope was estimated using the slope-fitting method described herein with reference to FIGS. 7, 8, and 9A-9P, above. Briefly, the ‘window’ method is a broadly applicable method that can robustly estimate the slope of the eCAP AGF for multiple patient populations and for various morphologies (linear and non-linear) of the eCAP AGF. This study included eCAP AGFs from three patient populations (children with CND, children with NSCNs, and adults) with various morphologies of the eCAP AGF. Therefore, the ‘window’ method was the most appropriate method for calculating the slope of each eCAP AGF.

The ‘window’ method was implemented by first resampling the original eCAP AGF (i.e., the recorded data points) at 11 data points in order to handle missing data points or non-uniformly sampled data in the original AGF. Then, a series of eight linear regression analyses were performed on sequential subsets of four data points (i.e., sliding window linear regression). Finally, the maximum slope was selected from among all subsets of data points (i.e., windows).

Building on the analytical models for quantifying the overall functional status of the CN using supervised machine learning techniques based on results of electrophysiological measures of the eCAP as described in Appendix A, a new analytical model is developed to estimate the quality of the ENI at individual electrode locations for the present study.

Just like in the original models described in Appendix A, the input/predictor variables of this new analytical model were eCAP parameters derived from the eCAP RRF and eCAP AGF. These parameters included t0, the eCAP threshold, the slope of the eCAP AGF, and the N1 latency of the eCAP with the maximum amplitude. However, the original models combined these four eCAP parameters measured at three electrode locations (i.e., 12 input variables) to predict the overall functional status of the CN (i.e., 1 output variable per subject). In contrast, the new analytical model was applied at each electrode location (i.e., four input variables and 1 output variable per electrode tested). Specifically, the four eCAP parameters recorded at a single electrode were combined to predict the quality of the ENI at that specific electrode location. The output for the electrode-specific model was a value between 0 and 100, where 0 and 100 represented the poorest and best ENI across all participants and electrodes, respectively. This electrode-specific number between 0 and 100 was defined as the local ENI index.

The training dataset for training the model included eCAP measures from 23 children with CND and 29 children with NSCNs recorded at three electrode locations. This is the same dataset that was used to train the original models and is further described in Appendix A. In the present study, the eCAP parameters in the training dataset were grouped together independent of electrode position. This was a modification from the original models in order to create a model that was applicable for any electrode location tested, and not just the electrode locations included in the training dataset. In other words, the grouping of the training data together eliminated any bias in the new model parameters that could be present due to the electrode location at which the eCAPs were measured.

Before finding the model parameters, each eCAP parameter was standardized across all participants to eliminate any model bias due to differences in scale between the eCAP parameters. Specifically, each eCAP parameter measured for each participant was standardized according to

$$x = \frac{{x}^{′} - μ}{σ} \qquad ( 2 )$$

where x was a vector containing the normalized value for the eCAP parameter, x′ was a vector containing the non-normalized value for the eCAP parameter, and μ and σ were the mean and standard deviation of x′, respectively.

The four standardized eCAP parameters were then used to find the model parameters (i.e., coefficients) of a linear regression model that separated the quality of the local ENI between the children with CND and the children with NSCNs. Specifically, the four standardized eCAP parameters were the input variables (x₁, x₂, x₃, x₄) and the output variable (y) was determined by patient population, where y=0 for children with CND and y=1 for children with NSCNs. The model parameters (β₀, β₁, β₂, β₃, β₄) were found according to

$${min}_{β , {β}_{0}} [ \frac{1}{N} {\sum }_{i = 1}^{N} {( {β}^{T} {x}_{i} + {β}_{0} - {y}_{i} )}^{2} ] \qquad ( 3 )$$

where β=[β₁ β₂ β₃ β₄], x=[x₁ x₂ x₃ x₄], β₀ was the model intercept term, τ represents the vector transpose, and N=156 (52 participants×3 electrodes) was the number of observations in the training data set.

In the original models, three different machine learning algorithms (linear 273 regression, support vector machine regression, and logistic regression) were used to create estimates of the functional status of the CN (i.e., CN indices) for individual CI patients. Linear regression was chosen for the creation of the model in the present study because linear regression produced CN indices that had the highest correlation with speech perception scores in adult CI patients among the three machine learning algorithms (see Appendix A).

### B. Model Validation

The model was validated with eCAP results from the 18 children with NSCNs (Participants: S1-S18) and the 18 children with CND and measurable eCAPs (Participants: CND2-CND18 and CND23) who were included in the study reported in He et al. (2018). Details regarding the testing procedures, electrodes tested, eCAP results, and participants' demographic information are provided He et al. (2018). Briefly, eCAP measures were recorded at seven electrodes (typically electrodes 3, 6, 9, 12, 15, 18, and 21) for children with NSCNs. The number and location of electrodes tested varied considerably in children with CND due to variations in the number of electrodes with measurable eCAPs among this patient population. The electrodes tested for each participant are listed in Table 1 of He et al. (2018).

The recorded eCAP parameters from each electrode for each participant in the validation dataset were standardized using the means and standard deviations calculated with the training data according to Equation 2 prior to model prediction. The standardized eCAP data from each participant were then mapped through the model function to obtain a predicted output variable (yₚ) for each participant according to

yₚ=β^{T}x+β₀ (4)

where β=[β1 β2 β3 β4], x=[x1 x2 x3 x4], β0 was the model intercept term as before. Finally, the local ENI index was calculated for each participant and electrode by scaling the output variable into the interval [0, 100] according to

$$Local ENI index = 100 ⋆ \frac{{y}_{p} - {y}_{min}}{{y}_{max} - {y}_{min}} \qquad ( 5 )$$

where yₘᵢₙ and yₘₐₓ were the minimum and maximum predicted output variables from the training dataset, respectively.

### C. Model Application

The validated model was then used to test the study hypotheses with the eCAP data from the adult study participants. As before with the validation dataset, the eCAP data were standardized according to Equation 2, and the local ENI index was calculated according to Equations 4 and 5. This process was repeated for each electrode listed in Table 1 resulting in a local ENI index corresponding to each electrode tested for each participant.

### D. Statistical Analysis

All statistical modeling and analysis for this study was performed using MATLAB (v. 2019b) software (Mathworks Inc., Natick, Mass., USA). The trust-region-reflective algorithm was used to estimate parameters of the mathematical functions used in statistical modeling. Each eCAP parameter was compared between children with CND and children with NSCNs in the training dataset with an unpaired, two-sample Welch's t-test. Welch's t-test was used because of unequal variances in the eCAP parameters between those two patient populations. As an initial comparison between children with CND and children with NSCNs in the validation dataset, an one-tailed, unpaired, two-sample t-test was used to compare the local ENI indices between these two groups, independent of the participant number and electrode location. For the adult study participants, the effects of study group and electrode location on each eCAP parameter and on local ENI index were assessed using generalized linear mixed effects models (GLMMs), with study group and electrode location as fixed effects and participant as a random effect. Pairwise comparisons of significant effects were evaluated using Tukey's honest significant difference (HSD). The effect of advanced age on the quality of local ENI was quantified with the slope obtained by linear regression with local ENI index as the dependent variable and age at testing as the independent variable.

### E. Results

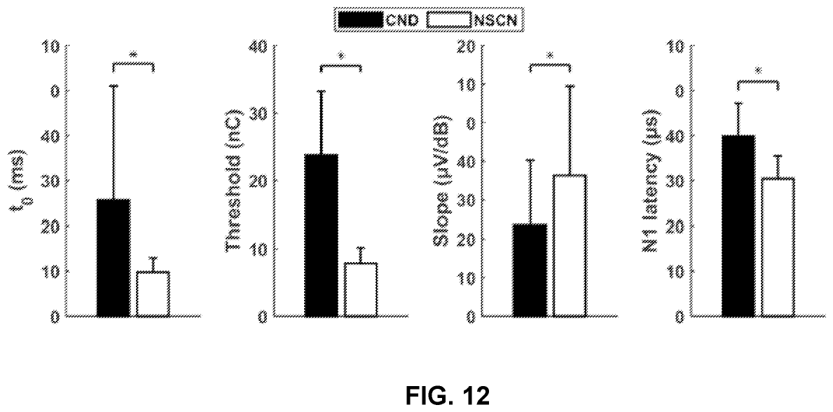

The means and standard deviations of eCAP parameters used in the model from both participant groups in the training dataset are shown in FIG. 12. As observed in the figure, the CND group had larger to and eCAP threshold values, smaller slopes of the eCAP AGF, and longer N1 latencies than the NSCN group. Results from unpaired two-sample Welch's t-tests confirm this result and showed significant differences between the two groups for all four eCAP parameters [t₀: t(72.7)=5.3, p<0.001; threshold: t(77.7)=14.0, p<0.001; slope: t(165.7)=−5.1, p<0.001; N1 latency: t(119.6)=9.3, p<0.001].

The coefficients for each model parameter in the predictive model are provided in Table 2. When excluding the model intercept term (β₀), the coefficient β₂ had a magnitude greater than two times the magnitude of any of the other coefficients (β₁, β₃, or β₄).

TABLE 2

| eCAP | Model | Model |
|---|---|---|
| parameter | parameter | coefficient |
| | β₀ | 0.571 |
| t₀ | β₁ | −0.041 |
| Threshold | β₂ | −0.287 |
| Slope | β₃ | 0.038 |
| N1 latency | β₄ | −0.110 |

Coefficients for each model parameter in the predictive model listed by corresponding eCAP parameter

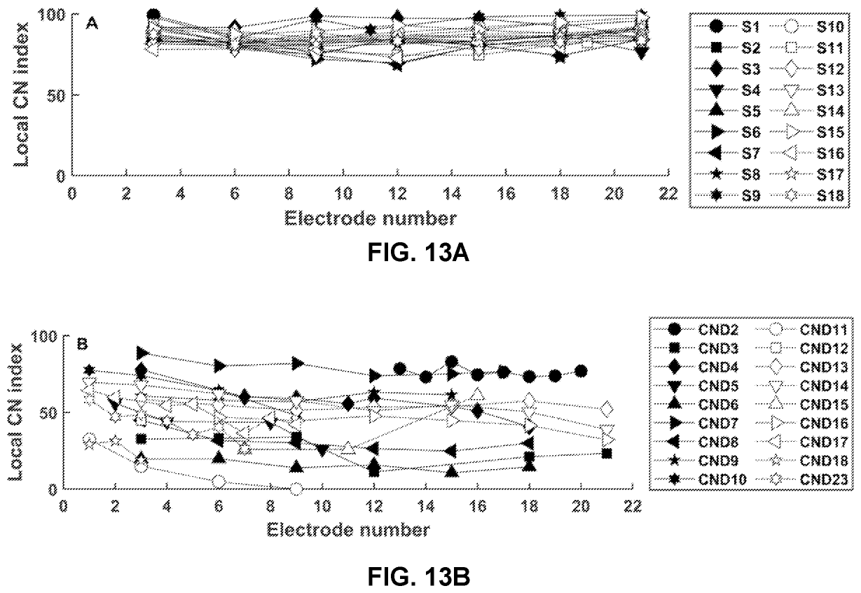

The local ENI index at each electrode tested for each participant in the validation dataset is shown in FIGS. 13A and 13B. It can be observed that the children with NSCNs generally had larger local ENI indices than children with CND at all seven electrode locations tested. Results of a one-tailed, unpaired, two-sample t-test that compared all of the local ENI indices between these two groups confirmed this observation [t(217)=19.1, p<0.001]. The children with NSCNs also had much smaller variation in local ENI indices across participants and electrode locations than the CND group. This is shown by the flat lines close to each other in FIG. 13A. On average, the local ENI index at the most apical electrode tested was greater than the local ENI index at the most basal electrode by only 0.94 (SD 6.4) in children with NSCNs.

In contrast, there was much more variability in local ENI indices across participants and electrodes for the children with CND. Specifically, some children had large local ENI indices at all electrodes tested that were within the range of the children with NSCNs (e.g., CND2 and CND7), while other children had very small local ENI indices (e.g., CND6 358 and CND11). There was also a general trend of decreasing local ENI index as the electrode location moved from the base to the apex of the cochlea. For the children with CND, 15 out of 17 (88%) participants had a smaller local ENI index at the most apical electrode tested than at the most basal electrode location. On average, the local ENI index at the most apical electrode tested was smaller than the local ENI index at the most basal electrode by 14.9 (SD 17.6). Participant CND12 was not included in these calculations of the change in local ENI index over electrode locations because only one electrode had a measurable eCAP.

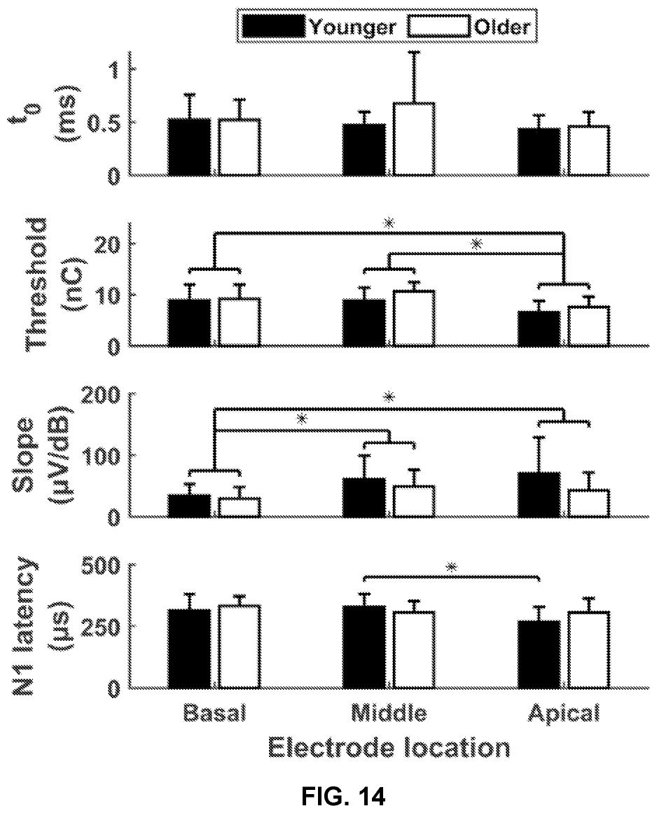

The means and standard deviations of eCAP parameters used for calculating local ENI indices for the adult study groups recorded at three electrode locations are shown in FIG. 14. As observed in the figure, the older adults on average had higher t0 and eCAP threshold values and smaller slopes of the eCAP AGF at all three electrode locations. However, results from the GLMMs indicated that there were no significant group differences for any of the eCAP parameters tested [t0: F(1,84)=1.52, p=0.221; eCAP threshold: F(1,84)=2.91, p=0.09; slope of eCAP AGF: F(1,84)=2.23, p=0.139; N1 latency: F(1,84)=0.68, p=0.412].

Another trend observed in FIG. 14 is that both study groups have lower eCAP thresholds and greater slopes at the apical electrode location than at the basal and middle electrode locations. Results from the GLMMs confirm an overall electrode effect on eCAP threshold [F(2,84)=13.58, p<0.001] and slope [F(2,84)=10.19, p<0.001]. There was also a significant effect of electrode location on N1 latency [F(2,84)=5.59, p=0.005], as well as a significant interaction between study group and electrode location [F(2,84)=3.37, p=0.039]. No other interactions were significant for any of the other eCAP parameters (F(2,84)≤2.15, p≥0.123). There was also not a significant effect of electrode location on t0 [F(2,84)=2.93, p=0.059]. The results from the GLMMs for each eCAP parameter, as well as significant post hoc comparisons, are provided in Table 3.

TABLE 3

| | Main effects | | Interaction effect |
|---|---|---|---|
| | Group | Electrode | Group × Electrode |
| t₀ | F_{(1,84)} = 1.52, p = 0.221 | F_{(2,84)} = 2.93, p = 0.059 | F_{(2,84)} = 2.15, p = 0.123 |
| eCAP threshold | F_{(1,84)} = 2.91, p = 0.091 | F_{(2,84)} = 13.58, p < 0.001 | F_{(2,84)} = 0.98, p = 0.378 |
| | | B > A, p = 0.008 | |
| | | M > A, p = 0.002 | |
| Slope of | F_{(1,84)} = 2.23, p = 0.139 | F_{(2,84)} = 10.19, p < 0.001 | F_{(2,84)} = 1.90, p = 0.157 |
| eCAP AGF | | B < M, p = 0.032 | |
| | | B < A, p = 0.021 | |
| N1 latency | F_{(1,84)} = 0.68, p = 0.412 | F_{(2,84)} = 5.59, p = 0.005 | F_{(2,84)} = 3.37, p = 0.039 |
| | | M > A, p = 0.026^{#} | |
| Local ENI index | F_{(1,84)} = 4.05, p = 0.047 | F_{(2,84)} = 1.56, p = 0.216 | F_{(2,84)} = 0.32, p = 0.724 |

Results from GLMMs and significant post hoc comparisons for each eCAP parameter and the local ENI index. GLMM: generalized linear mixed-effects model; eCAP: electrically-evoked Compound Action Potential; ENI: electrode-neuron interface; AGF: amplitude growth function; B: basal electrode location; M: middle electrode location; A: apical electrode location; #: post hoc comparisons are for the younger study group due to a significant interaction effect and nonsignificant post hoc comparisons for the older study group.

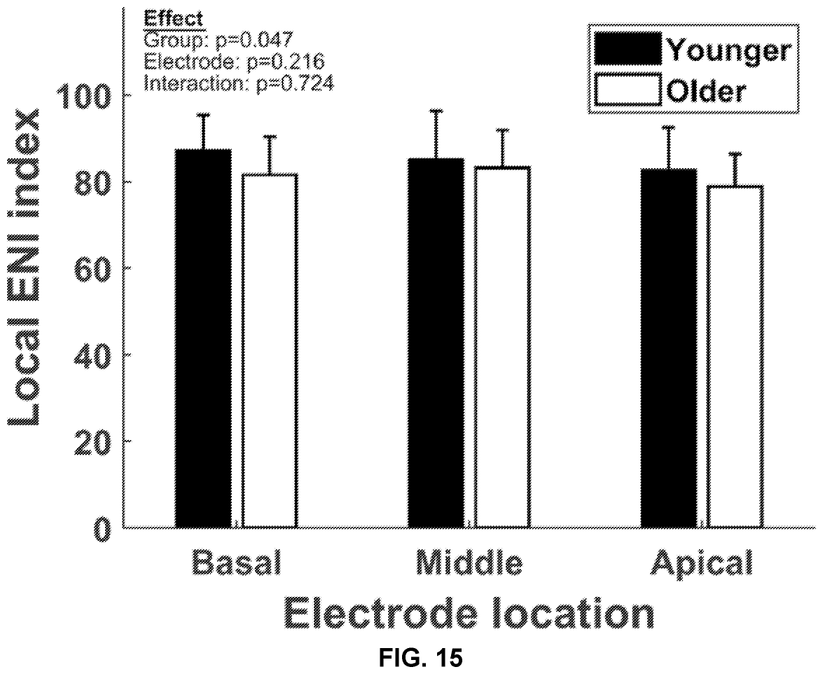

The means and standard deviations of the local ENI index calculated for both study groups at three electrode locations are shown in FIG. 15. As observed in the figure, the older adults on average had smaller local ENI indices at all three electrode locations. Results from the GLMMs confirmed this observation and indicated a significant group effect [F(1,84)=4.05, p=0.047]. There was not a significant effect of electrode location [F(2,84)=1.56, p=0.216] or a significant interaction [F(2,84)=0.32, p=0.724].

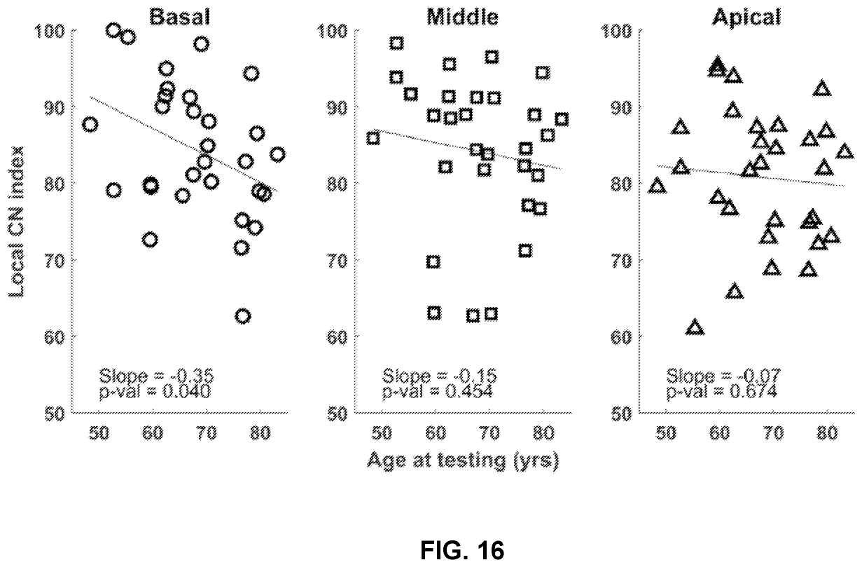

FIG. 16 shows the results of local ENI indices as a function of age at testing calculated at three electrode locations for all adults who participated in the study. Results of linear regression analyses are also included in each panel. Overall, the slope of the regression line ranged from −0.07 to −0.35, indicating a trend of decreasing local ENI index with increasing age. Also, the magnitude of the slope of the regression line decreased as the stimulating electrode moved from the base to the apex of the cochlea, indicating that the greatest effect of age on local ENI index occurred in the basal region of the cochlea. Results from the linear regression analyses indicated that the slope of the regression line was significantly different from zero for the electrode located in the basal region (p=0.040), but not for the electrodes located in the middle and apical regions of the cochlea (p=0.454 and p=0.674, respectively). The significant slope of −0.35 at the basal electrode implies a decrease of 3.5% in the quality of the ENI in the basal region of the cochlea per decade of increased age.

### F. Discussion of Determining Effects of Advanced Age on the Electrode-Neuron Interface

The relative magnitude of standardized regression coefficients can be used as a measure of the importance of each input variable in predicting the output variable (Mehmood et al., 2010). Therefore, the magnitudes of the model parameters that scale the eCAP parameters (i.e., β₁-β₄) represent the relative importance of each eCAP parameter in creating the local ENI index. As seen in Table 2, the regression coefficient for the eCAP threshold (i.e., β₂) had the highest magnitude among all other regression coefficients (excluding the offset term β₀) by at least double. This suggests that the eCAP 421 threshold is an important indicator for the quality of the ENI.

This expectation is supported by a recent study with CI patients that showed linear increases in eCAP thresholds with increased electrode distance from the mid-modiolar axis (MMA) and the medial wall (Schvartz-Leyzac et al., 2020). In the study, Schvartz-Leyzac et al. (2020) presented a linear model with the electrode-to-MMA distance which suggested that the eCAP threshold increased by 1.47 dB for every 1-mm increase in MMA distance. Similarly, Long et al. (2014) presented a linear model that suggested an average increase of 11 dB in psychophysical detection threshold for every 1-mm increase in electrode-to-modiolus distance. Cohen et al. (2001) also showed increased psychophysical detection thresholds with increased electrode-to-modiolus distance in a study with three CI patients. Collectively, these studies suggest that eCAP threshold is an important indicator of the quality of the ENI, specifically related to the position of the electrode in relation to the targeted CN fibers.

The local ENI index was validated with eCAP results from children with NSCNs and children with CND. It was expected that children with NSCNs would have greater local ENI indices than children with CND. The validation results followed that expectation. Specifically, there was a significant group difference in local ENI indices. Moreover, a much larger range of local ENI indices were generated for children with CND compared to children with NSCNs (FIG. 13). The local ENI index for children with CND ranged from very poor to high quality. This result agrees with studies that have reported outcomes for children with CND that range from no awareness of environmental sounds to open-set speech (Young et al., 2012; Vincenti et al., 2014; Birman et al., 2016; Han et al., 2019). Additionally, there was a general trend for children with CND to have smaller local ENI indices in the apical region than in the basal region on the cochlea. This general trend is expected because the cochlea forms in a base-to-apex sequence and CND is likely caused by prematurely halted inner ear development during embryogenesis (Jackler et 448 al., 1987). Therefore, the quality of the ENI should generally decrease along the length of the cochlea, with the level of damage related to when the inner ear stopped developing. This expectation is also supported by the result that the percentage of electrodes with recordable eCAP responses declined in children with CND as the testing electrode moved from the base to the apex of the cochlea (He et al., 2018). In contrast, eCAPs were recorded at all electrode locations for all children with NSCNs. Therefore, CI patients with fewer electrodes with recordable eCAPs would also be expected to have quickly declining quality of ENI as the test electrode moved toward more apical regions of the cochlea. This expectation is supported by the present results in which the children with CND who had few electrodes with recordable eCAPs also had sharp declines in local ENI index as the electrode tested moved more apically (FIG. 13). In contrast, the children with NSCNs and the children with CND who had recordable eCAPs along the length of the electrode array also had local ENI indices that did not change as dramatically with the change in electrode tested.

### G. Effect of Advanced Age on Individual eCAP Parameters and Local ENI Index

Results show that there was not a significant difference in absolute refractory periods, eCAP thresholds, slopes of eCAP AGFs, or N1 latencies between adults younger than 68 years and adults older than 68 years (Table 3). These results appear to be inconsistent with results that have demonstrated that children and young adults have steeper eCAP AGF linear slopes and higher eCAP thresholds than older individuals (Hughes et al., 2001; Cafarelli Dees et al., 2005; Brown et al., 2010; Jahn & Arenberg, 2020). However, these differences in age groups may be reflective of differences in etiology between patients who are pre-lingually deafened vs post-lingually deafened (Bodmer et al., 2007; Brown et al., 2010; Zarowski et al., 2020).

Interestingly, individual eCAP parameters by themselves have not been consistently shown to be correlated with speech perception scores. This non-significant association has been reported for the eCAP threshold (Brown et al., 1990; Cosetti et al., 2010; Franck & Norton, 2001; Kiefer et al., 2001; Turner et al., 2002; El Shennawy et al., 2015), the maximum eCAP amplitude (Brown et al., 1990), and slope of the eCAP AGF (Brown et al., 1990; Gantz et al., 1994; Franck & Norton, 2001; Turner et al., 2002; Kim et al., 2010). However, a significant group effect is observed when these eCAP parameters are combined by the predictive model to form the local ENI index (Table 3). These results suggest that individual eCAP parameters may not have sufficient predictive power by themselves, but do in combination.

Results also show that there was a significant effect of advanced age on the local ENI index in the basal region, but not in the middle and apical regions of the cochlea (FIG. 16). This result agrees with a recent histological study with human temporal bones in which the loss of CN fibers with age was significantly higher in the basal region than in the apical region of the cochlea (Wu et al., 2019). A different study with human temporal bones showed slightly more age-related degeneration of cell bodies of SGNs in the basal region than in the apical region of the cochlea (Makary et al., 2011). However, the difference in rates of degeneration of SGN cell bodies between regions of the cochlea did not reach a level of significance. This apparent discrepancy may be explained by the observation that CN fibers are lost almost three times faster than SGN cell bodies or inner hair cells (Wu et al., 2019).

### H. Clinical Application

Deactivating electrodes with poor ENIs from the clinical programming map may improve speech perception for CI patients by reducing interactions between channels and only stimulating highly-functional regions of the cochlea. Indeed, several studies have shown improvements in speech perception when excluding electrodes based on CT-imaging techniques (Noble et al., 2016), modulation detection thresholds (Garadat et al. 2013), detection thresholds (Zhou, 2017), and electrode discrimination scores (Zwolan et al., 1997; Saleh et al., 2013). However, other studies did not show a group difference in speech perception scores when excluding electrodes based on electrode discrimination scores (Vickers et al., 2016), detection thresholds (Bierer & Litvak, 2016) or the magnitude of the polarity effect (cathodic- vs anodic-dominant triphasic pulse) on detection thresholds (Goehring et al., 2019). Reasons for the inconsistency in results between these studies remain unknown, but may be influenced by differences in the number of electrodes excluded, the tests used to evaluate speech performance, and the criteria used to determine poor ENI.

The disclosed details a model that generated an index for the quality of the ENI at individual electrode locations by optimally combining four electrophysiological measures of the eCAP. While not explored in the present study, previous studies showed that a single index of overall CN function (i.e., CN index), created with machine learning algorithms and multiple measures derived from the eCAP AGF and eCAP RRF, was significantly correlated with speech perception in quiet (see Appendix A). In contrast, individual eCAP parameters have not been able to predict speech perception scores (Brown et al., 1990; Cosetti et al., 2010; Franck & Norton, 2001; Kiefer et al., 2001; Turner et al., 2002; El Shennawy et al., 2015). Therefore, deactivating electrodes in a programming map based on a combination of multiple factors, such as was done in this study, may be more successful than deactivating electrodes based on a single factor.

### I. Conclusions

The quality of the ENI at individual electrode locations can be quantified using a local ENI index generated using the newly developed analytical model. The new model demonstrates that the quality of the ENI declines with advanced age, especially in the basal region of the cochlea. Therefore, age-related decline in the quality of the ENI may contribute to speech perception deficits observed in older CI patients.

### IV. Computing Environment

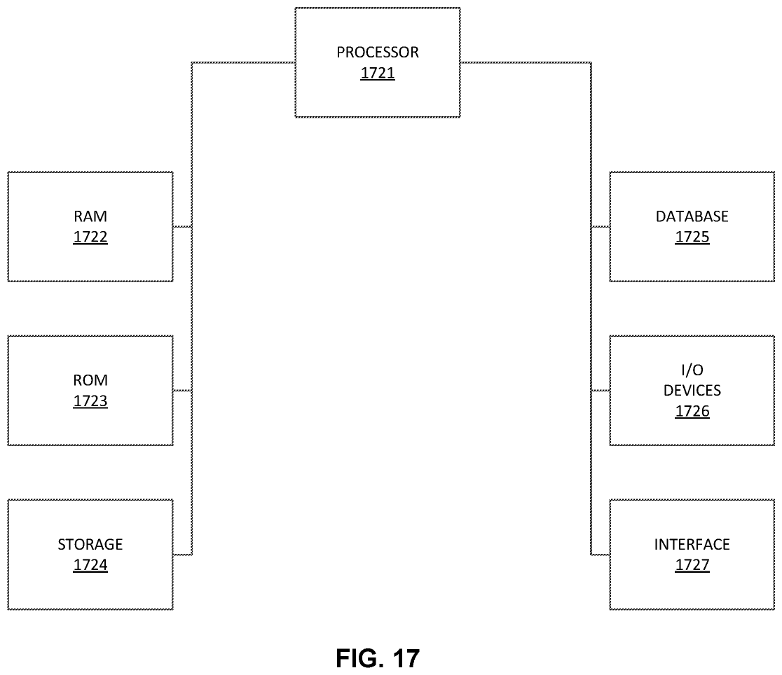

The above-described methods may be implemented on a computing system. The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software for methods of determining if a response is an eCAP, refining raw data of an eCAP AGF and utilizing maximum (i.e., steepest) slope from moving linear regression to effectively estimate cochlear nerve function, and determining quality of an ENI using a model developed from eCAP attributes. In one exemplary aspect, the units can comprise a computing device that comprises a processor 1721 as illustrated in FIG. 17 and described below.

FIG. 17 illustrates an exemplary computer that can be used to determine if a response is an eCAP, refine raw data of an eCAP AGF and utilizing maximum (i.e., steepest) slope from moving linear regression to effectively estimate cochlear nerve function, and determine quality of an ENI using a model developed from eCAP attributes. As used herein, “computer” may include a plurality of computers. The computers may include one or more hardware components such as, for example, a processor 1721, a random access memory (RAM) module 1722, a read-only memory (ROM) module 1723, a storage 1724, a database 1725, one or more input/output (I/O) devices 1726, and an interface 1727. All of the hardware components listed above may not be necessary to practice the methods described herein. Alternatively and/or additionally, the computer may include one or more software components such as, for example, a computer-readable medium including computer executable instructions for performing a method associated with the exemplary embodiments. It is contemplated that one or more of the hardware components listed above may be implemented using software. For example, storage 1724 may include a software partition associated with one or more other hardware components. It is understood that the components listed above are exemplary only and not intended to be limiting.

Processor 1721 may include one or more processors, each configured to execute instructions and process data to perform one or more functions associated with a computer for discriminating tissue of a specimen. Processor 1721 may be communicatively coupled to RAM 1722, ROM 1723, storage 1724, database 1725, I/O devices 1726, and interface 1727. Processor 1721 may be configured to execute sequences of computer program instructions to perform various processes. The computer program instructions may be loaded into RAM 1722 for execution by processor 1721.

RAM 1722 and ROM 1723 may each include one or more devices for storing information associated with operation of processor 1721. For example, ROM 423 may include a memory device configured to access and store information associated with the computer, including information for identifying, initializing, and monitoring the operation of one or more components and subsystems. RAM 1722 may include a memory device for storing data associated with one or more operations of processor 1721. For example, ROM 1723 may load instructions into RAM 1722 for execution by processor 1721.

Storage 1724 may include any type of mass storage device configured to store information that processor 1721 may need to perform processes consistent with the disclosed embodiments. For example, storage 1724 may include one or more magnetic and/or optical disk devices, such as hard drives, CD-ROMs, DVD-ROMs, or any other type of mass media device.

Database 1725 may include one or more software and/or hardware components that cooperate to store, organize, sort, filter, and/or arrange data used by the computer and/or processor 1721. For example, database 1725 may store raw data, as described herein and computer-executable instructions for determining if a response is an eCAP, refining raw data of an eCAP AGF and utilizing maximum (i.e., steepest) slope from moving linear regression to effectively estimate cochlear nerve function, and determining quality of an ENI using a model developed from eCAP attributes. It is contemplated that database 1725 may store additional and/or different information than that listed above.

I/O devices 1726 may include one or more components configured to communicate information with a user associated with computer. For example, I/O devices may include a console with an integrated keyboard and mouse to allow a user to maintain a database of digital images, results of the analysis of the digital images, metrics, and the like. I/O devices 1726 may also include a display including a graphical user interface (GUI) for outputting information on a monitor. I/O devices 1726 may also include peripheral devices such as, for example, a printer for printing information associated with the computer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 1727 may include one or more components configured to transmit and receive data via a communication network, such as the Internet, a local area network, a workstation peer-to-peer network, a direct link network, a wireless network, or any other suitable communication platform. For example, interface 1727 may include one or more modulators, demodulators, multiplexers, demultiplexers, network communication devices, wireless devices, antennas, modems, and any other type of device configured to enable data communication via a communication network.

### V. Conclusion

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain. The publications incorporated by reference include, but are not limited to, the following:
A. Abbas, P. J., Hughes, M., Brown, C. J., et al. (2004). Channel Interaction in Cochlear Implant Users Evaluated Using the Electrically Evoked Compound Action Potential. 548 Audiol Neurootol. 9. 203-13.
B. Bierer, J. A. (2010). Probing the electrode-neuron interface with focused cochlear implant stimulation. Trends Amplif, 14, 84-95.
C. Bierer, J. A., & Litvak, L. (2016). Reducing channel interaction through cochlear implant programming may improve speech perception: Current focusing and channel deactivation. Trends Hear, 20, 1-12.
D. Birman, C. S., Powell, H. R., Gibson, W. P., et al. (2016). Cochlear implant outcomes in cochlea nerve aplasia and hypoplasia. Otol Neurotol, 37, 438-445.
E. Bodmer, D., Shipp, D. B., Ostroff, J. M., et al. (2007). A comparison of postcochlear implantation speech scores in an adult population. Laryngoscope, 117(8), 1408-1411.
F. Botros, A., & Psarros, C. (2010). Neural Response Telemetry Reconsidered: II. The Influence of Neural Population on the eCAP Recovery Function and Refractoriness. Ear Hear, 31, 380-391.
G. Brown, C. J., Abbas, P. J., Gantz, B. (1990). Electrically evoked whole-nerve action potentials: data from human cochlear implant users. J Acoust Soc Am, 88, 1385-1391.
H. Brown, C. J., Abbas, P. J., Etler, C. P., O'Brien, S., & Oleson, J. J. (2010). Effects of long-term use of a cochlear implant on the electrically evoked compound action potential. J Am Acad Audiol, 21(1), 5-15.
I. Budenz, C. L., Cosetti, M. K., Coelho, D. H., et al. (2011). The effects of cochlear implantation on speech perception in older adults. J Am Geriatr Soc, 59, 446-453.
J. Cafarelli Dees, D., Dillier, N., Lai, W. K., et al. (2005). Normative findings of electrically evoked compound action potential measurements using the neural response telemetry of the Nucleus CI24M cochlear implant system. Audiol Neurootol, 10, 105-116.
K. Cohen, L. T., Saunders, E., & Clark, G. M. (2001). Psychophysics of a prototype peri-modiolar cochlear implant electrode array. Hear Res, 155(1-2), 63-81.
L. Cosetti, M. K., Shapiro, W. H., Green, J. E., et al. (2010). Intraoperative neural response 574 telemetry as a predictor of performance. Otol Neurotol. 31:1095-1099.
M. Eisen, M. D., & Franck, K. H. (2004). Electrically evoked compound action potential amplitude growth functions and HiResolution programming levels in pediatric CII implant subjects. Ear Hear, 25, 528-538.
N. El Shennawy, A. M., Mashaly, M. M., Shabana, M. I., Sheta, S. M. (2015). Telemetry changes over time in cochlear implant patients. Hearing Balance Commun. 13:24-31.
O. Franck, K. H., Norton, S. J. (2001). Estimation of psychophysical levels using the electrically evoked compound action potential measured with the neural response telemetry capabilities of Cochlear Corporation's CI24M device. Ear Hear. 22:289-299.
P. Friedland, D. R., Runge-Samuelson, C., Baig, H., et al. (2010). Case-control analysis of cochlear implant performance in elderly patients. Arch Otolaryngol Head Neck Surg, 136, 432-438.
Q. Gantz, B. J., Brown, C. J., Abbas, P. J. (1994). Intraoperative measures of electrically evoked auditory nerve compound action potential. Am J Otol. 15:137-144.
R. Garadat, S. N., Zwolan, T. A., & Pfingst, B. E. (2013). Using temporal modulation sensitivity to select stimulation sites for processor MAPs in cochlear implant listeners. Audiol Neurotol, 18(4), 247-260.
S. Goehring, T., Archer-Boyd, A., Deeks, J. M., et al. (2019). A site-selection strategy based on polarity sensitivity for cochlear implants: effects on spectro-temporal resolution and speech perception. J Assoc Res Otolaryngol, 20(4), 431-448.
T. Han, J. J., Suh, M. W., Park, M. K., et al. (2019). A Predictive Model for Cochlear Implant Outcome in Children with Cochlear Nerve Deficiency. Sci Rep, 9(1), 1154.
U. He, S., Shahsavarani, B. S., McFayden, T. C., et al. (2018). Responsiveness of the electrically stimulated cochlear nerve in children with cochlear nerve deficiency. Ear Hear, 39, 238-250.
V Hughes, M. L., Vander Werff, K. R., Brown, C. J., et al. (2001). A longitudinal study of electrode impedance, the electrically evoked compound action potential, and behavioral measures in nucleus 24 cochlear implant users. Ear hear, 22(6), 471-486.
W Jackler, R. K., Luxford, W. M., House, W. F. (1987). Congenital malformations of the inner ear: a classification based on embryogenesis. Laryngoscope, 97(3 Pt 2; Suppl 40), 2-14.
X. Jahn, K. N., & Arenberg, J. G. (2020). Electrophysiological Estimates of the Electrode-Neuron Interface Differ Between Younger and Older Listeners with Cochlear Implants. Ear Hear, 41(4), 948-960.
Y. Kiefer, J., Hohl, S., Sturzebecher, E., et al. (2001). Comparison of speech recognition with different speech coding strategies (SPEAK, CIS, and ACE) and their relationship to telemetric measures of compound action potentials in the nucleus CI 24M cochlear implant system. Audiology. 40:32-42.
Z. Kim, J. R., Abbas, P. J., Brown, C. J., et al. (2010). The relationship between electrically evoked compound action potential and speech perception: a study in cochlear implant users with short electrode array. Otol Neurotol, 31, 1041-1048.
AA. Kirby, A. E., & Middlebrooks, J. C. (2010). Auditory temporal acuity probed with cochlear implant stimulation and cortical recording. J Neurophysiol, 103(1), 531-542.
BB. Kirby, A. E., & Middlebrooks, J. C. (2012). Unanesthetized auditory cortex exhibits multiple codes for gaps in cochlear implant pulse trains. J Assoc Res Otolaryngol, 13(1), 67-80.
CC. Lenarz, M., Sonmez, H., Joseph, G., et al. (2012). Cochlear implant performance in geriatric patients. Laryngoscope, 122, 1361-1365.
DD. Lin, H. W., Furman, A. C., Kujawa, S. G., et al. (2011). Primary neural degeneration in the Guinea pig cochlea after reversible noise-induced threshold shift. J Assoc Res Otolaryngol, 12, 605-616.
EE. Lin, F. R., Chien, W. W., Li, L., et al. (2012). Cochlear implantation in older adults. Medicine, 91(5), 229.
FF. Long, C. J., Holden, T. A., McClelland, G. H., et al. (2014). Examining the electro-neural interface of cochlear implant users using psychophysics, CT scans, and speech understanding. J Assoc Res Otolaryngol, 15(2), 293-304.
GG. Makary, C. A., Shin, J., Kujawa, S. G., et al. (2011) Age-Related Primary Cochlear Neuronal Degeneration in Human Temporal Bones. JARO 12, 711-717.
HH. Mehmood, T., Liland, K. H., Snipen, L., et al. (2012). A review of variable selection methods in Partial Least Squares Regression. Chemometrics and Intelligent Laboratory Systems, 118, 62-69.
II. Miller, C. A., Abbas, P. J., Brown, C. J. (2000). An Improved Method of Reducing Stimulus Artifact in the Electrically Evoked Whole-Nerve Potential. Ear Hear, 21, 280-290.
JJ. Morsnowski, A., Charasse, B., Collet, L., et al. (2006). Measuring the Refractoriness of the Electrically Stimulated Auditory Nerve. Audiol Neurootol, 11, 389-402.
KK. Noble, J. H., Hedley-Williams, A. J., Sunderhaus, L., et al. (2016). Initial results with image-guided cochlear implant programming in children. Otol Neurotol, 37, e63-e69.
LL. Pfingst, B. E., Colesa, D. J., Watts, M. M., et al. (2017). Neurotrophin gene therapy in deafened ears with cochlear implants: long-term effects on nerve survival and functional measures. J Assoc Res Otolaryngol, 18, 731-750.
MM. Pfingst, B. E., Hughes, A. P., Colesa, D. J., et al. (2015). Insertion trauma and recovery of function after cochlear implantation: evidence from objective functional measures. Hear Res, 330, 98-105.
NN. Ramekers, D., Versnel, H., Strahl, S. B., et al. (2014). Auditory-nerve response to varied inter-phase gap and phase duration of the electric pulse stimulus as predicators for neuronal degeneration. J Assoc Res Otolaryngol, 15, 187-202.
OO. Roberts, D. S., Lin, H. W., Herrmann, B. S., et al. (2013). Differential cochlear implant outcomes in older adults. Laryngoscope, 123, 1952-1956.
PP. Saleh, S. M., Saeed, S. R., Meerton, L., et al. (2013). Clinical use of electrode differentiation to enhance programming of cochlear implants. Cochlear implants int, 14(sup4), 16-18.
QQ. Schvartz-Leyzac, K. C., & Pfingst, B. E. (2016). Across-site patterns of electrically evoked compound action potential amplitude-growth functions in multichannel cochlear implant recipients and the effects of the interphase gap. Hear Res, 341, 50-65.
RR. Schvartz-Leyzac, K. C., & Pfingst, B. E. (2018). Assessing the relationship between the electrically evoked compound action potential and speech recognition abilities in bilateral cochlear implant recipients. Ear Hear, 39, 344-358.
SS. Schvartz-Leyzac, K. C., Holden, T. A., Zwolan, T. A., et al. (2020). Effects of electrode location on estimates of neural health in humans with cochlear implants. J Assoc Res Otolaryngol, 21(3), 259-275.
TT. Shepherd, R. K., Roberts, L. A., Paolini, A. G. (2004). Long-term sensorineural hearing loss induces functional changes in the rat auditory nerve. Eur J Neurosci, 20, 3131-3140.
UU. Skidmore, J., Xu, L., Chao, X., et al. (2021a). Prediction of the Functional Status of the Cochlear Nerve in Individual Cochlear Implant Users Using Machine Learning and Electrophysiological Measures. Ear Hear. 42(1), 180-192.
VV. Skidmore, J., Ramekers, D., Colesa, D., et al. (2021b). A Robust and Broadly Applicable Method for Characterizing the Slope of the Electrically-evoked Compound Action Potential Amplitude Growth Function. Association for Research in Otolaryngology (ARO) 44th MidWinter Meeting.
WW. Sladen, D. P., & Zappler, A. (2015). Older and younger adult cochlear implant users: Speech recognition in quiet and noise, quality of life, and music perception. Am J Audiol, 24(1), 31-39.
XX. Turner, C., Mehr, M., Hughes, M., et al. (2002). Within-subject predictors of speech recognition in cochlear implants: a null result. Acoust Res Lett Online. 3:95-100.
YY. Vickers, D., Degun, A., Canas, A., et al. (2016). Deactivating cochlear implant electrodes based on pitch information for users of the ACE strategy. In Physiology, psychoacoustics and cognition in normal and impaired hearing (pp. 115-123). Springer, Cham.
ZZ. Vincenti, V., Ormitti, F., Ventura, E., et al. (2014). Cochlear implantation in children with cochlear nerve deficiency. Int J Pediatr Otorhinolaryngol, 78, 912-917.
AAA. Wiemes, G. R. M., Hamerschmidt, R., Moreira, A. T. R., et al. (2016). Auditory Nerve Recovery Function in Cochlear Implant Surgery with Local Anesthesia and Sedation versus General Anesthesia. Audiol Neurootol, 21, 150-157.
BBB. Wu, P. Z., Liberman, L. D., Bennett, K., et al. (2019). Primary Neural Degeneration in the Human Cochlea: Evidence for Hidden Hearing Loss in the Aging Ear. Neuroscience, 407, 8-20.
CCC. Young, N. M., Kim, F. M., Ryan, M. E., et al. (2012). Pediatric cochlear implantation of children with eighth nerve deficiency. Int J Pediatr Otorhinolaryngol, 76, 1442-1448.
DDD. Young, N. M., & Grohne, K. M. (2001). Comparison of pediatric Clarion recipients with and without the electrode positioner. Otol Neurotol, 22, 195-199.
EEE. Zhou, N. (2017). Deactivating stimulation sites based on low-rate thresholds improves spectral ripple and speech reception thresholds in cochlear implant users. J Acoust Soc Am, 141, EL243.
FFF. Zarowski, A., Molisz, A., De Coninck, L., et al. (2020). Influence of the pre- or postlingual status of cochlear implant recipients on behavioural T/C-levels. Int J Pediatr Otorhinolaryngol, 131, 109867.
GGG. Zekveld, A., Kramer, S. E., & Festen, J. (2011). Cognitive load during speech perception in noise: the influence of age, hearing loss, and cognition on pupil response. Ear Hear, 32, 498-510.
HHH. Zwolan, T. A., Collins, L. M., & Wakefield, G. H. (1997). Electrode discrimination and speech recognition in postlingually deafened adult cochlear implant subjects. J Acoust Soc Am. 102, 3673-3685.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method of determining whether an electrically evoked compound action potential (eCAP) exists in a neural response comprising:
providing a template eCAP waveform;
receive a recorded neural response waveform obtained from a patient with a cochlear implant;
re-sampling the recorded neural response waveform;
normalizing the re-sampled neural response waveform and the template eCAP waveform by subtracting mean voltages recorded in a first time period from the template eCAP waveform and the re-sampled neural response waveform;
determining a first negative (N1) peak and a trailing positive peak (P2) in each of the re-sampled neural response waveform and the template eCAP waveform;
scaling the template eCAP waveform vertically (voltage) to match the N1 and P2 amplitudes from the re-sampled neural response waveform and horizontally (time) to match the N1 and P2 latencies from the re-sampled neural response waveform;
trimming any portion of the scaled re-sampled neural response waveform and the scaled template eCAP waveform to a time period where both waveforms overlap;
re-sampling both the scaled re-sampled neural response waveform and the scaled template eCAP waveform at the same time periods as each other with higher resolution sampling occurring before the first time period to place emphasis on a first part of the waveforms in a correlation analysis;
calculating a correlation between the re-sampled scaled re-sampled neural response waveform and the re-sampled scaled template eCAP waveform; and
determining whether the correlation between the re-sampled scaled re-sampled neural response waveform and the re-sampled scaled template eCAP waveform indicates that the recorded neural response waveform comprises an eCAP.

2. (canceled)

3. The method of claim 1, wherein the neural response waveform is obtained by sending a user-defined stimuli through one electrode of the cochlear implant to stimulate surrounding neurons and recording an electrical response of the surrounding electrons using a neighboring electrode in the cochlear implant.

4. The method of claim 1, wherein re-sampling the recorded neural response waveform comprises up-sampling the recorded neural response waveform via cubic spline interpolation to create a smooth re-sampled neural response waveform at a higher effective sampling rate.

5. The method of claim 1, wherein subtracting mean voltages recorded in the first time period from the template eCAP waveform and the re-sampled neural response waveform comprises subtracting mean voltages recorded in the first 600 μ-sec from the template eCAP waveform and the re-sampled neural response waveform.

6. The method of claim 1, wherein re-sampling both the scaled re-sampled neural response waveform and the scaled template eCAP waveform at the same time periods as each other with higher resolution sampling occurring before the first time period to place emphasis on a first part of the waveforms in a correlation analysis comprises re-sampling both waveforms with higher resolution sampling occurring before 600 μ-sec to place emphasis on the first part of the waveforms in a correlation analysis.

7. The method of claim 1, wherein calculating the correlation between the re-sampled scaled re-sampled neural response waveform and the re-sampled scaled template eCAP waveform comprises calculating a Pearson correlation between the re-sampled scaled re-sampled neural response waveform and the re-sampled scaled template eCAP waveform.

8. (canceled)

9. A system for determining whether an electrically evoked compound action potential (eCAP) exists in a neural response comprising:
a memory; and
a processor in communication with the memory, wherein the processor executes computer-executable instructions stored in the memory, said instructions causing the processor to:
retrieve a template eCAP waveform from the memory;
receive a recorded neural response waveform obtained from a patient with a cochlear implant;
re-sample the recorded neural response waveform;
normalize the re-sampled neural response waveform and the template eCAP waveform by subtracting mean voltages recorded in a first time period from the template eCAP waveform and the re-sampled neural response waveform;
determine a first negative (N1) peak and a trailing positive peak (P2) in each of the re-sampled neural response waveform and the template eCAP waveform;
scale the template eCAP waveform vertically (voltage) to match the N1 and P2 amplitudes from the re-sampled neural response waveform and horizontally (time) to match the N1 and P2 latencies from the re-sampled neural response waveform;
trim any portion of the scaled re-sampled neural response waveform and the scaled template eCAP waveform to a time period where both waveforms overlap;
re-sample both the scaled re-sampled neural response waveform and the scaled template eCAP waveform at the same time periods as each other with higher resolution sampling occurring before the first time period to place emphasis on a first part of the waveforms in a correlation analysis;
calculate a correlation between the re-sampled scaled re-sampled neural response waveform and the re-sampled scaled template eCAP waveform; and
determine whether the correlation between the re-sampled scaled re-sampled neural response waveform and the re-sampled scaled template eCAP waveform indicates that the recorded neural response waveform comprises an eCAP.

10. (canceled)

11. The system of claim 9, wherein the neural response waveform is obtained by sending a user-defined stimuli through one electrode of the cochlear implant to stimulate surrounding neurons and recording an electrical response of the surrounding electrons using a neighboring electrode in the cochlear implant.

12. The system of claim 9, wherein re-sampling the recorded neural response waveform comprises up-sampling the recorded neural response waveform via cubic spline interpolation to create a smooth re-sampled neural response waveform at a higher effective sampling rate.

13. The system of claim 9, wherein subtracting mean voltages recorded in the first time period from the template eCAP waveform and the re-sampled neural response waveform comprises subtracting mean voltages recorded in the first 600 μ-sec from the template eCAP waveform and the re-sampled neural response waveform.

14. The system of claim 9, wherein re-sampling both the scaled re-sampled neural response waveform and the scaled template eCAP waveform at the same time periods as each other with higher resolution sampling occurring before the first time period to place emphasis on a first part of the waveforms in a correlation analysis comprises re-sampling both waveforms with higher resolution sampling occurring before 600 μ-sec to place emphasis on the first part of the waveforms in a correlation analysis.

15. The system of claim 9, wherein calculating the correlation between the re-sampled scaled re-sampled neural response waveform and the re-sampled scaled template eCAP waveform comprises calculating a Pearson correlation between the re-sampled scaled re-sampled neural response waveform and the re-sampled scaled template eCAP waveform.

16. (canceled)

17. A computer-program product comprising computer-executable instructions stored on a non-transitory medium, said computer-executable instructions for performing a method of determining whether an electrically evoked compound action potential (eCAP) exists in a neural response, said method comprising:
receiving a template eCAP waveform;
receive a recorded neural response waveform obtained from a patient with a cochlear implant;
re-sampling the recorded neural response waveform;
normalizing the re-sampled neural response waveform and the template eCAP waveform by subtracting mean voltages recorded in a first time period from the template eCAP waveform and the re-sampled neural response waveform;
determining a first negative (N1) peak and a trailing positive peak (P2) in each of the re-sampled neural response waveform and the template eCAP waveform;
scaling the template eCAP waveform vertically (voltage) to match the N1 and P2 amplitudes from the re-sampled neural response waveform and horizontally (time) to match the N1 and P2 latencies from the re-sampled neural response waveform;
trimming any portion of the scaled re-sampled neural response waveform and the scaled template eCAP waveform to a time period where both waveforms overlap;
re-sampling both the scaled re-sampled neural response waveform and the scaled template eCAP waveform at the same time periods as each other with higher resolution sampling occurring before the first time period to place emphasis on a first part of the waveforms in a correlation analysis;
calculating a correlation between the re-sampled scaled re-sampled neural response waveform and the re-sampled scaled template eCAP waveform; and
determining whether the correlation between the re-sampled scaled re-sampled neural response waveform and the re-sampled scaled template eCAP waveform indicates that the recorded neural response waveform comprises an eCAP.

18. (canceled)

19. The computer-program product of claim 17, wherein the neural response waveform is obtained by sending a user-defined stimuli through one electrode of the cochlear implant to stimulate surrounding neurons and recording an electrical response of the surrounding electrons using a neighboring electrode in the cochlear implant.

20. The computer-program product of claim 17, wherein re-sampling the recorded neural response waveform comprises up-sampling the recorded neural response waveform via cubic spline interpolation to create a smooth re-sampled neural response waveform at a higher effective sampling rate.

21. The computer-program product of claim 17, wherein subtracting mean voltages recorded in the first time period from the template eCAP waveform and the re-sampled neural response waveform comprises subtracting mean voltages recorded in the first 600 μ-sec from the template eCAP waveform and the re-sampled neural response waveform.

22. The computer-program product of claim 17, wherein re-sampling both the scaled re-sampled neural response waveform and the scaled template eCAP waveform at the same time periods as each other with higher resolution sampling occurring before the first time period to place emphasis on a first part of the waveforms in a correlation analysis comprises re-sampling both waveforms with higher resolution sampling occurring before 600 μ-sec to place emphasis on the first part of the waveforms in a correlation analysis.

23. The computer-program product of claim 17, wherein calculating the correlation between the re-sampled scaled re-sampled neural response waveform and the re-sampled scaled template eCAP waveform comprises calculating a Pearson correlation between the re-sampled scaled re-sampled neural response waveform and the re-sampled scaled template eCAP waveform.

24. (canceled)

25. A method of refining raw data of an electrically evoked compound action potential (eCAP) amplitude growth function (AGF) and utilizing maximum (i.e., steepest) slope from moving linear regression to effectively estimate cochlear nerve function, wherein the maximum slope provides an estimate for the slope for the raw AGF and correlates that slope with an estimated number of surviving neurons in the cochlear nerve.

26. The method of claim 25, wherein the method comprises:
1) receiving raw data comprised of a plurality of data points of AGF data;
2) resampling the raw data into a plurality of linearly spaced data points of AGF data;
3) performing linear regression on a moving window comprised of a subset (N) of the plurality of linearly spaced data points of AGF data:
3) (a) perform linear regression on a first window of comprised of data points 1 to N of the plurality of linearly spaced data points of AGF data to determine a slope of the first window,
3) (b) move the window by one point to form a second window and perform linear regression on data points 2 to N+1 to determine a slope of this second window,
3) (c) continue to perform linear regression on the data points of the plurality of linearly spaced data points of AGF data using the same size subset (N) of data points until the end of the plurality of linearly spaced data points of AGF data is reached to determine a slope of each of the plurality of different moving windows;
4) determine the steepest (i.e., maximum) slope among the slopes calculated in the plurality of different windows; and
5) correlate the selected steepest slope with an estimate of surviving neurons in the cochlear nerve.

27. The method of claim 25, wherein the estimated number of surviving neurons in the cochlear nerve are used to provide cochlear implant patients with a better clinical experience including design, selection and/or specification of the cochlear implant as well as adjustment of the cochlear implant.

28. A system for refining raw data of an electrically evoked compound action potential (eCAP) amplitude growth function (AGF) and utilizing maximum (i.e., steepest) slope from moving linear regression to effectively estimate cochlear nerve function, wherein the maximum slope provides an estimate for the slope for the raw AGF and correlates that slope with an estimated number of surviving neurons in the cochlear nerve, said system comprising:
a memory; and
a processor in communication with the memory, wherein the processor executes computer-executable instructions stored in the memory, said instructions causing the processor to:
1) receive raw data comprised of a plurality of data points of AGF data;
2) resample the raw data into a plurality of linearly spaced data points of AGF data;
3) perform linear regression on a moving window comprised of a subset (N) of the plurality of linearly spaced data points of AGF data:
3) (a), perform linear regression on a first window of comprised of data points 1 to N of the plurality of linearly spaced data points of AGF data to determine a slope of the first window,
3) (b) move the window by one point to form a second window and perform linear regression on data points 2 to N+1 to determine a slope of this second window,
3) (c) continue to perform linear regression on the data points of the plurality of linearly spaced data points of AGF data using the same size subset (N) of data points until the end of the plurality of linearly spaced data points of AGF data is reached to determine a slope of each of the plurality of different moving windows;
4) determine the steepest (i.e., maximum) slope among the slopes calculated in the plurality of different windows; and
5) correlate the selected steepest slope with an estimate of surviving neurons in the cochlear nerve.

29. The system of claim 28, wherein the estimated number of surviving neurons in the cochlear nerve are used to provide cochlear implant patients with a better clinical experience including design, selection and/or specification of the cochlear implant as well as adjustment of the cochlear implant.

30. A computer-program product comprising computer-executable instructions stored on a non-transitory medium, said computer-executable instructions for performing A method of refining raw data of an electrically evoked compound action potential (eCAP) amplitude growth function (AGF) and utilizing maximum (i.e., steepest) slope from moving linear regression to effectively estimate cochlear nerve function, wherein the maximum slope provides an estimate for the slope for the raw AGF and correlates that slope with an estimated number of surviving neurons in the cochlear nerve, said method comprising:
1) receiving raw data comprised of a plurality of data points of AGF data;
2) resampling the raw data into a plurality of linearly spaced data points of AGF data;
3) performing linear regression on a moving window comprised of a subset (N) of the plurality of linearly spaced data points of AGF data:
3) (a) perform linear regression on a first window of comprised of data points 1 to N of the plurality of linearly spaced data points of AGF data to determine a slope of the first window,
3) (b) move the window by one point to form a second window and perform linear regression on data points 2 to N+1 to determine a slope of this second window,
3) (c) continue to perform linear regression on the data points of the plurality of linearly spaced data points of AGF data using the same size subset (N) of data points until the end of the plurality of linearly spaced data points of AGF data is reached to determine a slope of each of the plurality of different moving windows;
4) determine the steepest (i.e., maximum) slope among the slopes calculated in the plurality of different windows; and
5) correlate the selected steepest slope with an estimate of surviving neurons in the cochlear nerve.

31. The computer program product of claim 30, wherein the estimated number of surviving neurons in the cochlear nerve are used to provide cochlear implant patients with a better clinical experience including design, selection and/or specification of the cochlear implant as well as adjustment of the cochlear implant.

32. A method of determining a quality of an interface between an electrode of a cochlear implant and a neuron or group of neurons, said method comprising:
develop a model based on parameters of electrically evoked compound action potential (eCAP) attributes measured in individual test subjects; and
estimate a quality of an electrode-neuron interface (ENI) at an individual electrode location in a cochlear implant user using the developed model.

33. The method of claim 32, wherein the eCAP attributes include absolute refractory period, eCAP threshold, eCAP slope, and eCAP N1 latency.

34. The method of claim 32, wherein the test subjects are grouped into different age groups.

35. The method of claim 34, wherein the developed model is used to estimate effects of advanced age on the quality of the electrode-neuron interface.
